(19) United States
(12) Patent Application Publication
(10) Pub. No.: US 2024/0377321 A1
(43) Pub. Date: Nov. 14, 2024

(54) HOLOGRAPHIC IMAGE PROCESSING AND DATA EXTRACTION

(71) Applicant: Accelerate Diagnostics, Inc., Tucson, AZ (US)

(72) Inventors: Landon K Prisbrey, Tucson, AZ (US); Niels Oppermann, Hilo, HI (US); Austin Ashby, Tucson, AZ (US)

(21) Appl. No.: 18/316,502

(22) Filed: May 12, 2023

(51) Int. Cl. G01N 21/45; G01N 33/569; G03H 1/04

(52) U.S. Cl. CPC G01N 21/453; G01N 33/569; G03H 1/04

(57) ABSTRACT

Systems, methods, and computer program products for analyzing a sample volume. One or more holographs of the sample volume are generated. Each holograph includes a plurality of pixels, and each pixel has an intensity. Information is extracted from the holographs by analyzing the pixels to determine a property of the sample volume without first reconstructing photographs from the holograph. Methods of extracting the information include determining a dispersion factor for the intensity of pixels, and extracting holographic features from the plurality of pixels that belong to a class of shapes including one or more diffraction patterns.

### FIELD OF THE INVENTION

The present invention relates generally to holographic imaging and, more particularly, to holographic imaging of biological samples and extraction of data from the holographic images.

### BACKGROUND

Microbial infections are best treated as early as possible to provide the greatest opportunity for patient recovery and to limit morbidity and mortality. Roughly 85% of patients demonstrating symptoms of infection will not have sufficient microorganism concentrations in their blood at initial presentation to enable detection of the causative agent. Corresponding blood samples may appear negative for microorganisms until many doubling events occur, at which point enough microbial cells will be present to reach the lower threshold of standard detection testing.

Conventional automated microscopy systems for detecting microbial cells in patient samples comprise various configurations of sample containers, reaction reservoirs, reagents, and optical detection systems. These optical detection systems are configured to obtain images via dark field and fluorescence photomicrographs of microorganisms contained in reaction reservoirs such as flow cells, chambers, microfluidic channels, and the like. Such systems typically include a controller configured to direct operation of the system and process microorganism information derived from the photomicrographs. However, these systems are generally incapable of detecting low concentrations of microorganisms directly in patient specimens. They also require a culturing period to ensure that, if viable microbial cells are present, they reach a detectable level to statistically ensure that a negative reading is truly negative.

A phenotypical approach to detection of a viable microbial population in a sample involves in vitro monitoring of microbial growth. While many approaches have been proposed to achieve this, solutions based on direct optical interrogation remain elusive. Optical approaches are typically constrained by factors such as optical resolution as well as the need for timely acquisition of microbial growth over time. Detection of small concentrations of viable bacteria (e.g., less than 10⁵ colony-forming units per milliliter (CFU/mL)) presents additional challenges as it requires large volumes of patient specimens to be interrogated to ensure a high probability of detection.

Optical interrogation at high resolution typically relies on lengthy multiple pass scanning methods employing high precision three-dimensional stages, high quality objectives, and fine focusing techniques. Moreover, label-free bacteria require the use of less common imaging modes such as phase contrast or differential contrast interference microscopy due to small differences in refractive index with suspension media. As a result, hardware and software requirements for such applications scale poorly with the sample volume under investigation.

Thus, there is a need for improved systems, methods, and computer program products for quickly detecting and characterizing microbial cells in patient samples at early stages of infection.

### SUMMARY

In an embodiment of the invention, a sample analysis system is provided. The system includes a holographic imager configured to generate a holograph of a sample volume, one or more processors operatively coupled to the holographic imager, and a memory operatively coupled to the one or more processors that stores program code. When the program code is executed by the one or more processors, it causes the system to generate a first holograph of the sample volume at a first time that includes a first plurality of pixels each having an intensity, determine a first dispersion factor of the intensity of at least a first portion of the first plurality of pixels, and determine a property of the sample volume based on a value of the first dispersion factor.

In an aspect of the system, the program code may further cause the system to determine the property of the sample volume based on the value of the first dispersion factor by comparing the value of the first dispersion factor to a predetermined threshold value.

In another aspect of the system, the program code may further cause the system to generate a second holograph of the sample volume at a second time that includes a second plurality of pixels each having an intensity, determine a second dispersion factor of the intensity of at least a second portion of the second plurality of pixels, and determine the property of the sample volume based on the value of the first dispersion factor by comparing the value of the first dispersion factor to the value of the second dispersion factor.

In another aspect of the system, the first portion of the first plurality of pixels may be one of a plurality of portions of the first plurality of pixels, and the program code may further cause the system to determine a second dispersion factor of the intensity of a second portion of the first plurality of pixels, and determine the property of the sample volume based on the value of the first dispersion factor by comparing the first dispersion factor to the second dispersion factor.

In another aspect of the system, the program code may further cause the system to identify a portion of interest in the first plurality of portions, determine a z-height of an object generating a diffraction pattern in the portion of interest, and analyze the object.

In another aspect of the system, the program code may further cause the system to analyze the object by reconstructing a photograph from the first holograph at the z-height.

In another aspect of the system, the program code may cause the system to identify the portion of interest by determining a dispersion factor of the intensity of each portion of the first plurality of pixels to generate a plurality of dispersion factors, comparing the value of each dispersion factor of the plurality of dispersion factors to one or more values of other dispersion factors of the plurality of dispersion factors, and identifying the dispersion factor of the portion of interest as an outlier from the plurality of dispersion factors.

In another aspect of the system, each portion of the plurality of portions of the first plurality of pixels may provide a tile of a plurality of tiles of the first holograph.

In another aspect of the system, the program code may further cause the system to apply one or more image modification processes that do not involve image reconstruction to the first holograph prior to determining the first dispersion factor.

In another aspect of the system, the one or more image modification processes may include a flat-field correction process.

In another aspect of the system, the one or more image modification processes may include identifying one or more irrelevant portions of the first holograph that are not relevant to quantifying a change in the property of the sample volume, generating a mask configured to remove the one or more irrelevant portions of the first holograph, and applying the mask to the first holograph.

In another aspect of the system, the sample volume may include one or both of a plurality of microorganisms and a plurality of eukaryotic cells of animal or human origin.

In another aspect of the system, the plurality of microorganisms may belong to a species or class of Gram-negative bacteria, Gram-positive bacteria, or fungi.

In another aspect of the system, the first dispersion factor may be a variance.

In another embodiment of the invention, a method for analyzing the sample volume it presented. The method includes generating the first holograph of the sample volume at the first time that includes the first plurality of pixels each having an intensity, determining the first dispersion factor of the intensity of at least the first portion of the first plurality of pixels, and determining the property of the sample volume based on the value of the first dispersion factor.

In another aspect of the method, determining the property of the sample volume based on the value of the first dispersion factor may include comparing the value of the first dispersion factor to the predetermined threshold value.

In another aspect of the method, the method may further include generating the second holograph of the sample volume at the second time including the second plurality of pixels each having an intensity, determining the second dispersion factor of the intensity of at least the second portion of the second plurality of pixels, and determining the property of the sample volume based on the value of the first dispersion factor by comparing the value of the first dispersion factor to the value of the second dispersion factor.

In another aspect of the method, the first portion of the first plurality of pixels may be one of the plurality of portions of the first plurality of pixels, and the method may further include determining the second dispersion factor of the intensity of the second portion of the first plurality of pixels, and determining the property of the sample volume based on the value of the first dispersion factor by comparing the first dispersion factor to the second dispersion factor.

In another aspect of the method, the method may further include identifying the portion of interest in the first plurality of portions, determining the z-height of the object generating the diffraction pattern in the portion of interest, and analyzing the object.

In another aspect of the method, analyzing the object may include reconstructing the photograph from the first holograph at the z-height.

In another aspect of the method, identifying the portion of interest may include determining the dispersion factor of the intensity of each portion of the first plurality of pixels to generate the plurality of dispersion factors, comparing the value of each dispersion factor of the plurality of dispersion factors to one or more values of other dispersion factors of the plurality of dispersion factors, and identifying the dispersion factor of the portion of interest as an outlier from the plurality of dispersion factors.

In another aspect of the method, each portion of the plurality of portions of the first plurality of pixels may provide a tile of the plurality of tiles of the first holograph.

In another aspect of the method, the method may further include applying the one or more image modification processes that do not involve image reconstruction to the first holograph prior to determining the first dispersion factor, wherein the one or more image modification processes do not involve image reconstruction.

In another aspect of the method, the one or more image modification processes may include the flat-field correction process.

In another aspect of the method, the one or more image modification processes may include identifying one or more irrelevant portions of the first holograph that are not relevant to quantifying a change in the property of the sample volume, generating the mask configured to remove the one or more irrelevant portions of the first holograph, and applying the mask to the first holograph.

In another aspect of the method, the sample volume may include one or both of the plurality of microorganisms and the plurality of eukaryotic cells of animal or human origin.

In another aspect of the method, the plurality of microorganisms may belong to the species or class of Gram-negative bacteria, Gram-positive bacteria, or fungi.

In another embodiment of the invention, a computer program product is provided. The computer program product includes a non-transitory computer-readable storage medium, and program code stored on the non-transitory computer-readable storage medium. The program code is configured so that, when executed by one or more processors, the program code causes the one or more processors to cause a holographic imager to generate the first holograph of the sample volume at the first time that includes the first plurality of pixels each having an intensity, determine the first dispersion factor of the intensity of at least the first portion of the first plurality of pixels, and determine the property of the sample volume based on the value of the first dispersion factor.

In another embodiment of the invention, another sample analysis system is provided. The system includes the holographic imager configured to generate the holograph of the sample volume, the one or more processors operatively coupled to the holographic imager, and the memory operatively coupled to the one or more processors that stores program code. When the program code is executed by the one or more processors, it causes the system to generate the first holograph of the sample volume at the first time that includes the first plurality of pixels each having an intensity, extract a first set of holographic features from at least a first portion of the first plurality of pixels that belong to a class of shapes including one or more diffraction patterns each associated with a diffraction of light by an object in the sample volume, determine a first number of holographic features in the first set of holographic features, and determine a property of the sample volume based on a value of the first number of holographic features.

In an aspect of the system, the program code may cause the system to determine the property of the sample volume based on the value of the first number of holographic features by comparing the value of the first number of holographic features to a predetermined threshold value.

In another aspect of the system, the program code may further cause the system to generate the second holograph of the sample volume including the second plurality of pixels each having an intensity at the second time, extract a second set of holographic features from at least a second portion of the second plurality of pixels that belong to the class of shapes including the one or more diffraction patterns, and determine a second number of holographic features in the second set of holographic features. In this aspect of the system, the program code may cause the system to determine the property of the sample volume based on the value of the first number of holographic features by comparing the value of the first number of holographic features to the value of the second number of holographic features.

In another aspect of the system, the class of shapes may include one or more patterns having a radial symmetry.

In another aspect of the system, the program code may further cause the system to determine a phase shift associated with light passing through the object in the sample volume.

In another aspect of the system, the program code may cause the system to determine the phase shift by fitting a mathematical formula to a first fringe pattern generated by the object in the first holograph, and extracting a parameter from the mathematical formula indicative of the phase shift.

In another aspect of the system, the phase shift of the object may be used to distinguish the object from one or more other objects having different phase shifts.

In another aspect of the system, the object may be a cell, and the one or more other objects may be debris.

In another aspect of the system, the object may be a first type of cell, and the one or more other objects may include a second type of cell.

In another aspect of the system, the first portion of the first plurality of pixels may be one of a plurality of portions of the first plurality of pixels, and the program code may further cause the system to extract a second set of holographic features from the second portion of the first plurality of pixels that belong to the class of shapes including the one or more diffraction patterns, determine a second number of holographic features in the second set of holographic features, and determine the property of the sample volume based on the value of the first number of holographic features by comparing the first number of holographic features to the second number of holographic features.

In another aspect of the system, the program code may further cause the system to identify a portion of interest in the plurality of portions of the first plurality of pixels, determine a z-height of the object generating the diffraction pattern in the portion of interest, and analyze the object.

In another aspect of the system, the program code may cause the system to analyze the object by reconstructing a photograph from the first holograph at the z-height.

In another aspect of the system, the program code may cause the system to identify the portion of interest by extracting a set of holographic features from each portion of the plurality of portions of the first plurality of pixels, determining a number of holographic features in each set of holographic features extracted from the plurality of portions, comparing the number of holographic features in each set of holographic features to the number of holographic features in the other sets of holographic features, and identifying the number of holographic features extracted from the portion of interest as an outlier from the number of holographic features in the other sets of holographic features.

In another aspect of the system, each portion of the plurality of portions of the first plurality of pixels may provide a tile of a plurality of tiles of the first holograph.

In another aspect of the system, the sample volume may include one or both of the plurality of microorganisms and the plurality of eukaryotic cells of animal or human origin.

In another aspect of the system, the plurality of microorganisms may belong to a species or class of Gram-negative bacteria, Gram-positive bacteria, or fungi.

In another embodiment of the invention, another method of analyzing the sample volume is presented. The method includes generating the first holograph of the sample volume at the first time that includes the first plurality of pixels each having an intensity, extracting the first set of holographic features from at least the first portion of the first plurality of pixels that belong to the class of shapes including one or more diffraction patterns each associated with the diffraction of light by the object in the sample volume, determining the first number of holographic features in the first set of holographic features, and determining the property of the sample volume based on the value of the first number of holographic features.

In an aspect of the method, determining the property of the sample volume based on the value of the first number of holographic features may include comparing the value of the first number of holographic features to the predetermined threshold value.

In another aspect of the method, the method may further include generating the second holograph of the sample volume at the second time including the second plurality of pixels each having an intensity, extracting the second set of holographic features from at least the second portion of the second plurality of pixels that belong to the class of shapes including the one or more diffraction patterns, and determining the second number of holographic features in the second set of holographic features.

In this aspect of the method, determining the property of the sample volume based on the value of the first number of holographic features may include comparing the value of the first number of holographic features to the value of the second number of holographic features.

In another aspect of the method, the class of shapes may include one or more patterns having a radial symmetry.

In another aspect of the method, the method may further include determining the phase shift associated with light passing through the object in the sample volume.

In another aspect of the method, determining the phase shift may include fitting the mathematical formula to the first fringe pattern generated by the object in the first holograph, and extracting a parameter from the mathematical formula indicative of the phase shift.

In another aspect of the method, the phase shift of the object may be used to distinguish the object from one or more other objects having different phase shifts.

In another aspect of the method, the object may be a cell, and the one or more other objects may be debris.

In another aspect of the method, the object may be the first type of cell, and the one or more other objects may include the second type of cell.

In another aspect of the method, the first portion of the first plurality of pixels may be one of the plurality of portions of the first plurality of pixels, and the method may further include extracting the second set of holographic features from the second portion of the first plurality of pixels that belong to the class of shapes including the one or more diffraction patterns, determining the second number of holographic features in the second set of holographic features, and determining the property of the sample volume based on the value of the first number of holographic features by comparing the first number of holographic features to the second number of holographic features.

In another aspect of the method, the method may further include identifying the portion of interest in the plurality of portions of the first plurality of pixels, determining the z-height of the object generating the diffraction pattern in the portion of interest, and analyzing the object.

In another aspect of the method, analyzing the object may include reconstructing the photograph from the first holograph at the z-height.

In another aspect of the method, identifying the portion of interest may include extracting a set of holographic features from each portion of the plurality of portions of the first plurality of pixels, determining the number of holographic features in each set of holographic features extracted from the plurality of portions, comparing the number of holographic features in each set of holographic features to the number of holographic features in the other sets of holographic features, and identifying the number of holographic features extracted from the portion of interest as an outlier from the number of holographic features in the other sets of holographic features.

In another aspect of the method, each portion of the plurality of portions of the first plurality of pixels may provide a tile of the plurality of tiles of the first holograph.

In another aspect of the method, the sample volume may include one or both of the plurality of microorganisms and the plurality of eukaryotic cells of animal or human origin.

In another aspect of the method, the plurality of microorganisms may belong to a species or class of Gram-negative bacteria, Gram-positive bacteria, or fungi.

In another embodiment of the invention, another computer program product is provided. The computer program product includes a non-transitory computer-readable storage medium, and program code stored on the non-transitory computer-readable storage medium. The program code is configured so that, when executed by the one or more processors, the program code causes the one or more processors to cause a holographic imager to generate the first holograph of the sample volume at the first time that includes the first plurality of pixels each having an intensity, extract the first set of holographic features from at least the first portion of the first plurality of pixels that belong to the class of shapes including one or more diffraction patterns each associated with the diffraction of light by the object in the sample volume, determine the first number of holographic features in the first set of holographic features, and determine the property of the sample volume based on the value of the first number of holographic features.

The above summary presents a simplified overview of some embodiments of the invention to provide a basic understanding of certain aspects of the invention discussed herein. The summary is not intended to provide an extensive overview of the invention, nor is it intended to identify any key or critical elements, or delineate the scope of the invention. The sole purpose of the summary is merely to present some concepts in a simplified form as an introduction to the detailed description presented below.

### BRIEF DESCRIPTION OF THE DRAWINGS

The accompanying drawings, which are incorporated in and constitute a part of this specification, illustrate various embodiments of the invention and, together with the general description of the invention given above, and the detailed description of the embodiments given below, serve to explain the embodiments of the invention.

FIG. 1 is a diagrammatic view of an exemplary holographic imager including a light source and an image sensor.

FIG. 2 is a diagrammatic view of a diffraction pattern generated by an object on a photosensitive surface of the imaging sensor of FIG. 1.

FIG. 3 is an image of an exemplary holograph that may be generated by the imaging sensor of FIG. 1 or 2.

FIG. 4 is a perspective view of an exemplary sample analysis system including a light source assembly and sensor assembly.

FIG. 5 is a front view of the sensor assembly of FIG. 4.

FIG. 6 is a front view of the light source assembly of FIG. 4.

FIGS. 7 and 8 are front views of the sensor assembly of FIG. 5 showing insertion of a microfluidic card configured to hold sample volumes.

FIG. 9 is a diagrammatic view depicting a process for reconstructing photographs from a holograph.

FIG. 10 is a diagrammatic view of an exemplary image flattening process for flattening a holograph.

FIG. 11 is a diagrammatic view of an exemplary image masking process for masking a holograph.

FIG. 12 is a diagrammatic view showing a sequence of holographs taken over a period of time and a sequence of photographs that were reconstructed from the holographs depicting cell growth in a sample volume.

FIG. 13 is a graphical view showing a plot of a variance in the intensity of the pixels in the holographs of FIG. 12 and a plot of a mean intensity of the pixels in the photographs of FIG. 12.

FIGS. 14 and 15 are graphical views including plots that compare using holograph intensity variance in a time series of holographs to characterize a sample volume versus using full image reconstruction from the time series of holographs.

FIG. 16 is a diagrammatic view showing a diffraction pattern of an individual object as captured by a holograph and a corresponding graph which shows a theoretical curve representing a fit to the empirical diffraction pattern.

FIG. 17 is a diagrammatic view of an object detection process for analyzing holographs.

FIGS. 18A-18E depict a sequence of tiled photographs of a sample volume experiencing cellular growth.

FIG. 19 is a graphical view including plots of a cellular growth indicator extracted from either the entire holograph or a single tile of the holograph of a sequence of holographs of the sample volume of FIGS. 18A-18E that show an earlier event detection based on the growth indicator extracted from the single tile.

FIG. 20 is a flowchart depicting a process for preparing a sequence of holographs for data extraction.

FIGS. 21 and 22 are diagrammatic views of holographs illustrating the process of FIG. 20.

FIG. 23 is a diagrammatic view of a computer that may be used to implement one or more of the components or processes shown in FIGS. 1-22.

It should be understood that the appended drawings are not necessarily to scale, and may present a somewhat simplified representation of various features illustrative of the basic principles of the invention. The specific design features of the sequence of operations disclosed herein, including, for example, specific dimensions, orientations, locations, and shapes of various illustrated components, may be determined in part by the particular intended application and use environment. Certain features of the illustrated embodiments may have been enlarged or distorted relative to others to facilitate visualization and a clear understanding. In particular, thin features may be thickened, for example, for clarity or illustration.

### DETAILED DESCRIPTION

Embodiments of the present invention are directed to systems and methods that use in-line holography to detect the presence of objects, such as microbial cells, which are suspended in a sample volume. In-line holography refers to a process that involves shining light through the sample volume to generate a diffraction pattern, and capturing an image of the diffraction pattern which is referred to herein as a “holograph”. The diffraction pattern is generated due to the diffraction of light by the objects, which define a three-dimensional suspension in the medium of the sample volume. An in-line holographic imaging system may include a light source that illuminates the sample volume, a sample holder configured to receive a consumable in the form of a sample container that contains the sample volume, and an image sensor that captures the holograph.

Conventional imaging techniques generate non-holographic images (referred to herein as “photographs”) by focusing light (e.g., with a lens) to form a focused image on a photosensitive surface of the image sensor. Photographic systems typically rely on capturing photographs of cells growing in each of a plurality of focal planes located in the sample volume. This requires repeated focusing and capturing of multiple photographs (e.g., one for each focal plane) at each of a plurality of selected sample times. In contrast, holographic imaging systems only need to capture one holograph of the sample volume at each of a plurality of selected sample times.

The sample volume may be analyzed using methods that avoid the need to focus on any single event in the sample volume. Algorithms may be used to extract information from one or more of the holographs, e.g., by using Fourier transformations to reconstruct a photograph of the objects in each of one or more focal planes. The focal planes for which reconstructed photographs are generated may be selected on the basis of a non-reconstructive analysis of the holograph. Three or four-dimensional holographic methods may also be used to extract data from one or more holographs of the sample volume. For example, reconstructed photographs across multiple focal planes of a sample volume (three dimensions) may be obtained over time (four dimensions) using video frame rates.

Several strategies may be employed to extract information from a holograph that is useful in determining the properties of the sample volume which generated the holograph. Reconstruction-based strategies include using a Fourier transformation to reconstruct a photograph of microscopic quality at any height (i.e., in any z-plane) in the sample volume. However, image reconstruction processes tend to be computationally intense, and use complex algorithms to not only perform the transformation, but to also find an appropriate data rich z-plane from which to reconstruct the photograph. This can become an iterative process, and thus poses a serious bottleneck when multiple holographs and cell incubations need to be analyzed simultaneously.

Direct-from-holograph strategies extract data from the holograph pertaining to a property of the sample volume without first reconstructing photographs from the holograph. Properties of the sample volume which may be determined include, for example, the number and/or characteristics of one or more objects suspended in the sample volume.

The holographic imaging systems and accompanying methods of capturing and analyzing holographs disclosed herein enable the analysis of objects in holographs without first reconstructing “real space” representations (e.g., photographs) of the sample volume using angular spectrum or similar image reconstruction techniques. A conventional understanding of holographs implies that the net signal or differential within each holograph is null due to the summation of waves emanating from object diffraction patterns in the holograph cancelling each other out. However, the disclosed direct-from-holograph methods of analysis overcome this theoretical limitation imposed by the wave-cancelling null hypothesis. Advantageously, the direct-from-holograph methods of analysis disclosed herein may also consume fewer computational resources as compared to conventional image reconstruction-based holograph analysis techniques.

In some cases, direct-from-holograph methods may provide enough information about a sample that image reconstruction becomes unnecessary. However, information extracted from the holograph using direct-from-holograph approaches may also be used to determine when and where (e.g., at what time and for which z-plane) to reconstruct a photograph. For example, object detection in one or more holographs may allow for targeted reconstruction of a photograph in specific regions of interest in the sample volume, thereby negating the need to reconstruct multiple photographs at different z-planes or even a full photograph at a single z-plane. Exemplary areas of interest may include object-rich regions of the sample volume where notable cell growth or morphology changes are taking place, e.g., due to being exposed to an effector. Information extracted from holographs may also be used to learn the precise geometry of the holography setup so that more advanced reconstruction approaches can be used. Specific direct-from-holograph methods which may be applied directly to the holograph, either singularly or in combination, are described in more detail below.

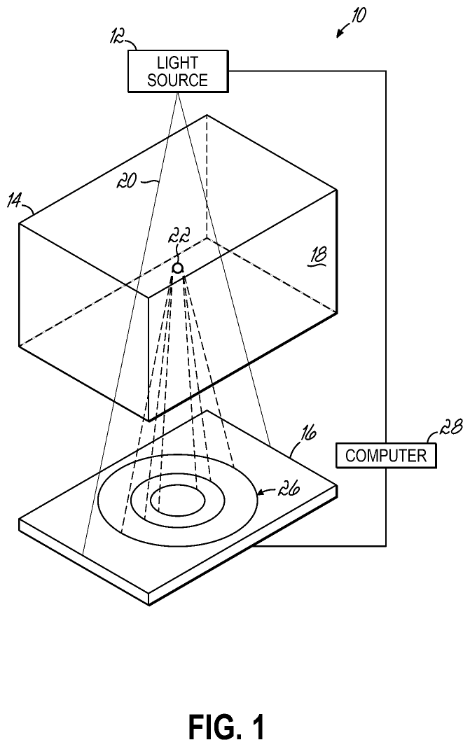

FIG. 1 depicts an exemplary holographic imager 10 including a light source 12, a sample container 14, and an image sensor 16. The light source 12 may include, for example, a laser diode or point source of light that illuminates a sample volume 18 contained in the sample container 14 with light 20, e.g., coherent light. The light source 12 may include lenses, filters, apertures, or other optical components (not shown) configured to modify the coherence of the light 20 received by the sample volume 18. The sample volume 18 may include an object 22 (e.g., a colony of bacteria) suspended in a medium (e.g., culture media and/or gelling agent). As the light 20 passes through the sample volume 18, a portion of the light 20 may be diffracted by the object 22. The diffracted portion of the light 20 may interact with other portions of the light 20 to generate a diffraction pattern 26 at the image sensor 16. The light source 12 and image sensor 16 may be operatively coupled to a computer 28. The computer 28 may be configured to cause the light source 12 to illuminate the sample volume 18 and the image sensor 16 to capture an image of the diffraction pattern 26 in the form of a holograph. The computer 28 may also store and/or analyze the holographs, as well as data extracted from the holographs.

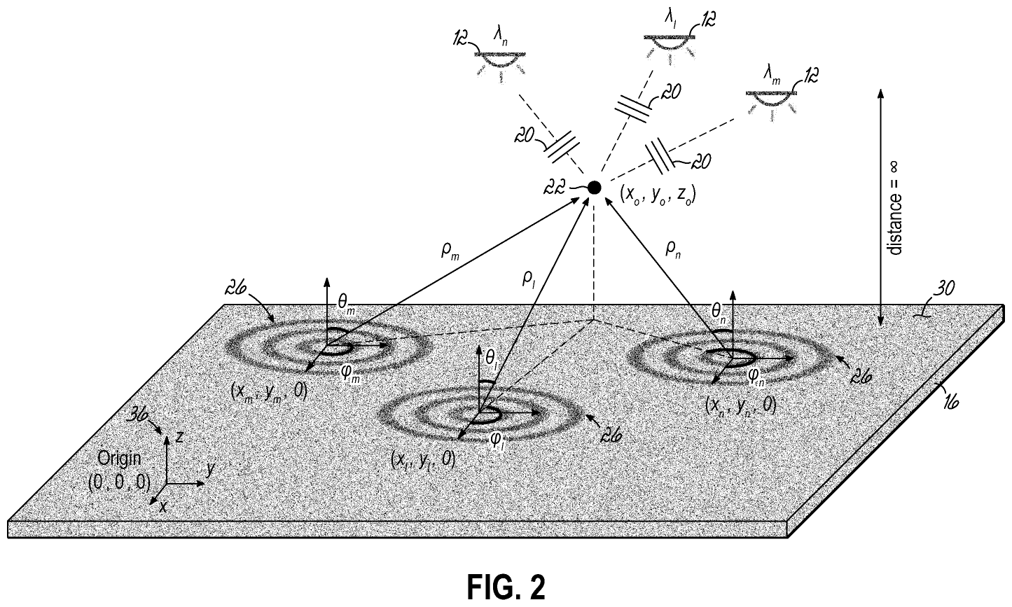

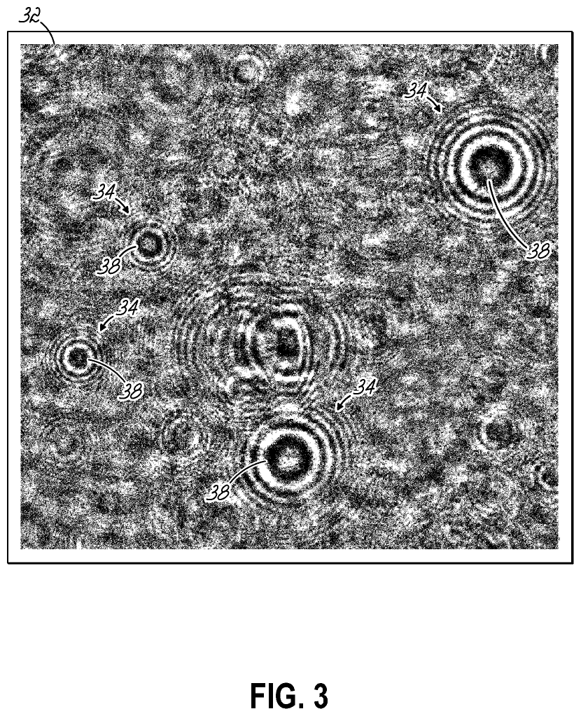

FIG. 2 depicts the generation of a diffraction pattern on a photosensitive surface of an image sensor 16, which defines a sensor plane 30, and FIG. 3 depicts an exemplary holograph 32 which may be generated by the image sensor 16. The holograph 32 illustrates a diffraction pattern that may be generated by a plurality of objects 22 suspended in the sample volume 18, and depicts what is essentially a superposition of the individual diffraction patterns 26 generated by each of the objects 22. Each of the circular patterns in the holograph 32 associated with the diffraction pattern 26 generated by a single object 22 may be referred to herein as a holographic feature 34 of the holograph 32. One or more holographs 32 may be generated by the image sensor 16 using a plurality of light sources 12, e.g., three laser diodes that emit light 20 (e.g., coherent light) at each of three different wavelengths λₗ, λₘ, λₙ. For embodiments in which the image sensor 16 can differentiate between different colors of light (e.g., a red, green, and blue color image sensor), different wavelengths of light may be used to facilitate analysis of the different diffraction patterns generated by the interaction of the object 22 with each individual light source 12. In an alternative embodiment, each light source 12 may emit light having the same spectral content, but be activated at different non-overlapping times so that a seperate holograph 32 can be captured by the image sensor 16 for each light source 12.

The position of a holographic feature 34 in the holograph 32 may be determined relative to a fixed reference frame 36 defined by set of unit-length direction vectors. The unit-length vectors of the reference frame 36 may include an x-axis and a y-axis orthogonal to the x-axis, with each of the x and y-axes being coplanar with the sensor plane 30, and thus the holograph 32 generated by the image sensor 16. By way of example, the x-axis may be parallel to a height dimension of the sensor plane 30, and the y-axis may be parallel to a width dimension of the sensor plane 30. A z-axis of reference frame 36 may be orthogonal to both the x and y-axes, and thus orthogonal to the sensor plane 30. The x, y, and z-axes may thereby form a right-handed coordinate system for defining the positions of objects 22 in the sample volume 18 and holographic features 34 on the holograph 32. The origin of the reference frame 36 may define a point with coordinates of (0,0,0), and may be located on the sensor plane 30. Accordingly, all coordinates on the sensor plane 30 have z-coordinate value z=0, and thus every point of the holograph 32 has a known z-coordinate value z=0.

The object 22 may be located between the light sources 12 and the image sensor 16 so that the object 22 generates diffraction patterns 26 on the photosensitive surface of image sensor 16. Each light source 12 may be positioned relative to the image sensor 16 so that the light 20 propagates at an angle having a different azimuth φ₁, φₘ, φₙ and/or elevation θ, θₘ, θₙ relative to the object 22 as compared to the other light sources 12. The position each holographic feature 34 may correspond to the position of the diffraction pattern 26 on the photosensitive surface of image sensor 16 that defined the holographic feature 34. Accordingly, each holographic feature 34 may have a different position in the holograph 32 that can be defined by the coordinates (xₗ, yₗ, 0), (xₘ, yₘ, 0), (xₙ, yₙ, 0) if its center 38 on the sensor plane 30. The coordinates of the holographic feature 34 on the holograph 32 may be used to determine the position of the object 22 that generated the diffraction pattern 26 associated with the holographic feature 34. The light sources 12 may be configured so that the light 20 has essentially plane waves at the object 22. Thus, each light source 12 may be treated as being at an infinite distance d from the object 22 for purposes of diffraction pattern analysis.

Each light source 12 may cause the object 22 to generate a diffraction pattern 26 at the image sensor 16 having a unique shape and position (x, y, 0), and each of these diffraction patterns 26 may be analyzed as a separate holograph 32. The number of light sources 12 and their placement may vary in different holographic imagers 10. In response to a diffraction pattern 26 changing over time, a sample analysis system may determine that the object 22 associated with the diffraction pattern 26 is changing, e.g., growing, shrinking, becoming more/less transparent, altering its shape, etc.

To increase throughput, sample analysis systems may have a plurality of image sensors 16 each configured to capture holographs 32 from a different sample volume 18. Multiple image sensors 16 may facilitate detection of changing object behavior over a short time period. By way of example, a holograph of each sample volume 18 may be obtained every 10, 20, or 30 minutes for a period of one to three hours. For sample volumes 18 including microorganisms, a one to three hour time period may provide enough time for two to three (or more) doublings of objects 22 in a growth chamber or flow cell.

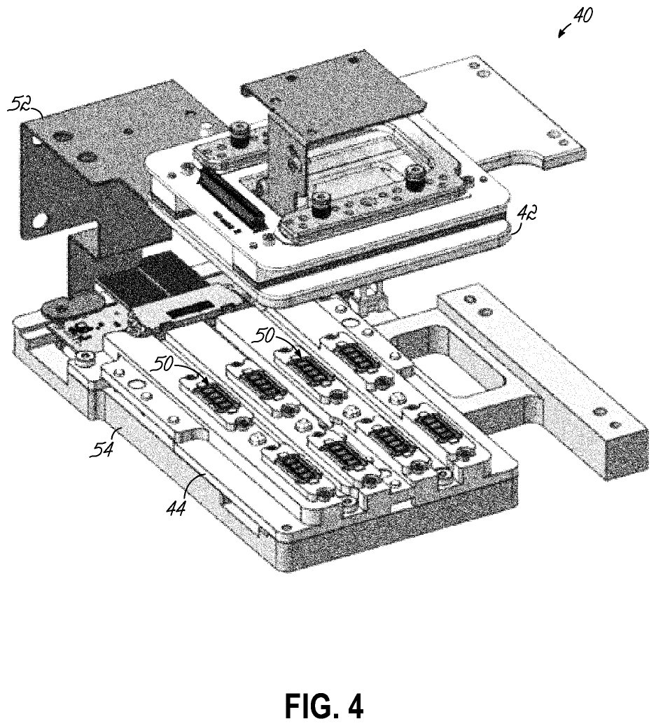

FIGS. 4-8 depict an exemplary sample analysis system 40 including a light source assembly 42 and an image sensor assembly 44. The image sensor assembly 44 may be configured to receive a microfluidic card 46 or other device that includes or is configured to receive a plurality of sample containers 14. When the microfluidic card 46 is inserted into image sensor assembly 44, each sample container 14 may be selectively positioned between the light source assembly 42 and the image sensor assembly 44 depending on the position of the microfluidic card 46 in the image sensor assembly 44.

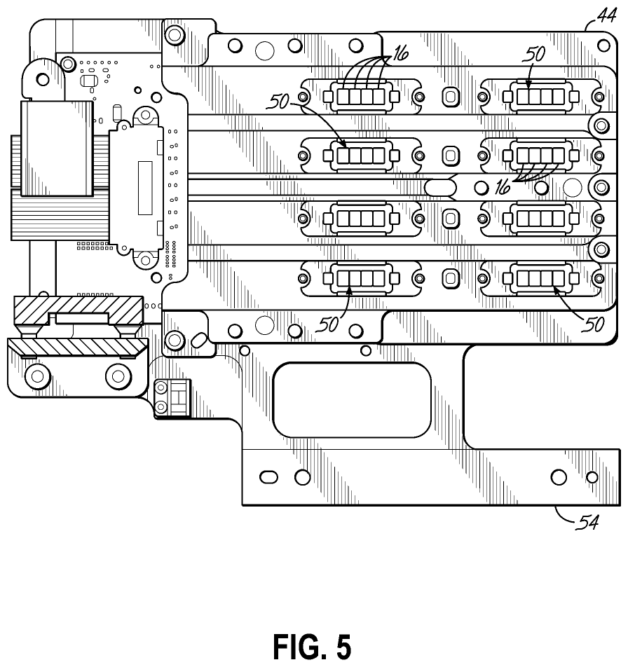

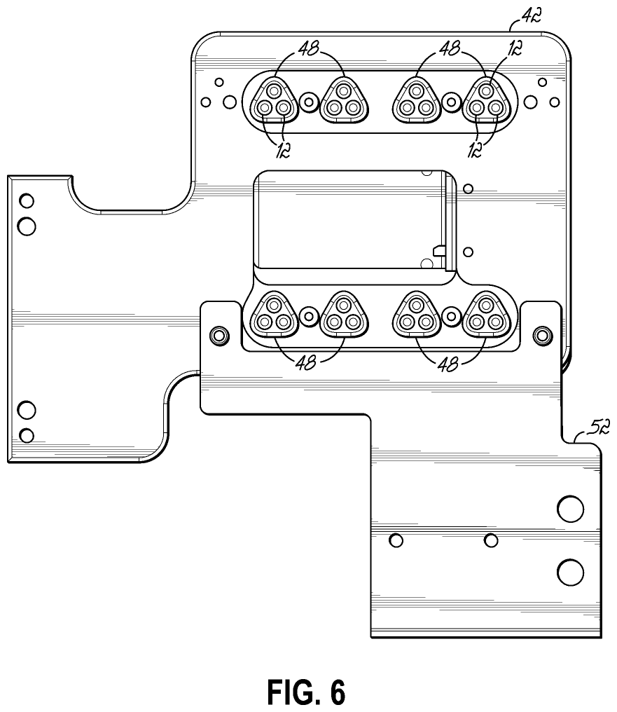

As best shown by FIG. 5, the image sensor assembly 44 may include one or more sensor arrays 50 each including one or more image sensors 16, e.g., eight sensor arrays 50 each including four image sensors 16. As best shown by FIG. 6, the light source assembly 42 may include a plurality of light source subassemblies 48 each including one or more light sources 12, e.g., eight light source subassemblies 48 each including three light sources 12.

The light source assembly 42 and image sensor assembly 44 may be configured so that each light source subassembly 48 is positioned opposite an associated sensor array 50 to collectively define a holographic imager 10. The light source assembly 42 may be positioned relative to the image sensor assembly 44 by a support bracket 52 that is operatively coupled to the image sensor assembly 44 through a sensor assembly base 54. Each holographic imager 10 may be configured to generate a holograph 32 of each of a plurality of sample volumes 18 at a time, e.g., one holograph 32 per sensor of the sensor array 50.

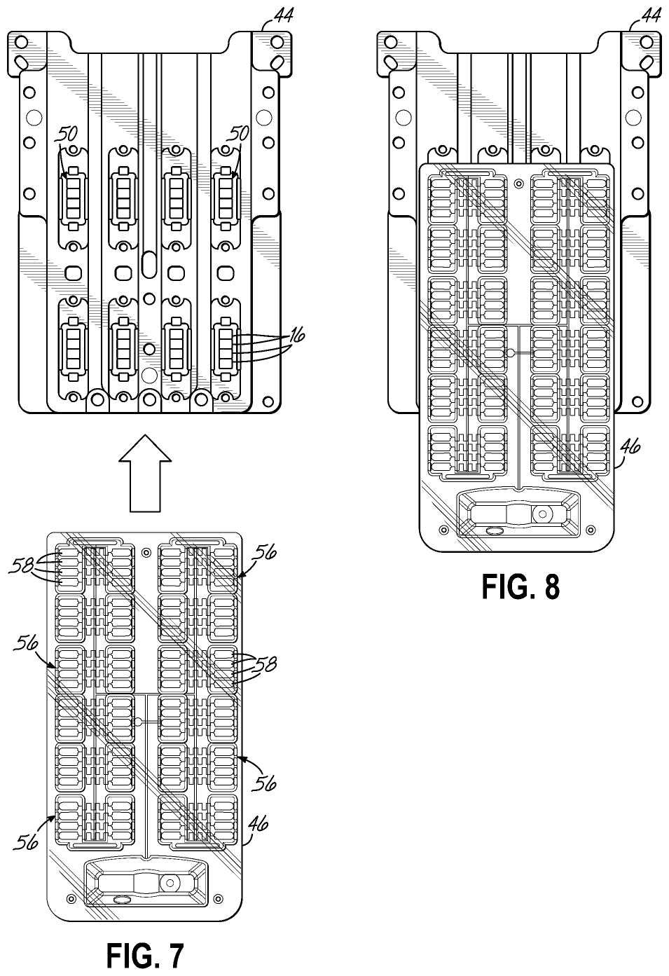

The microfluidic cards 46 may include a plurality of pods 56 each having a plurality of sample containers 14 in the form of wells 58, e.g., twelve pods 56 each having eight wells 58. Each sample container 14 may be configured to receive a sample volume 18. As best shown by FIGS. 7 and 8, the image sensor assembly 44 may be configured to receive microfluidic cards 46 in each of a plurality of predetermined positions, e.g., three positions. Each predetermined position may align a respective portion of the pods 56 (e.g., four pods 56) with the sensor arrays 50.

When light 20 emitted by light sources 12 encounters objects 22, the light waves may be distorted from their original path. A diffraction pattern 26 generated by the diffracted light may then be recorded by the image sensor 16 as a holograph 32. Each holographic imager 10 may include one or more image sensors 16 to monitor and capture events happening in multiple areas of one or more sample containers 14, e.g., chambers or flow cells. The exemplary sample analysis system 40 depicted by FIGS. 4-8 includes thirty two image sensors 16 arranged into eight sensor arrays 50 each having four image sensors 16, however embodiments are not limited to any particular number of image sensors or arrays. This configuration of image sensors 16 may be organized to accommodate an assay consumable, such as the depicted multi-well microfluidic card 46, whose chambers or flow cells provide a sample container 14 that is positioned above the sensor arrays 50. In the present example, the consumable is depicted as a 96-well microfluidic card 46 that is positioned directly above a sensor array 50. However, it should be understood that embodiments are not limited to consumables including any particular number or type of sample containers 14. In any case, the sensor array 50 may record events occurring in the sample containers 14 when the light sources 12 above the card emit a flash of light.

In-line holography relies on a coherent light source emitting light with a well-defined and predictable wavefront. One way of achieving this in practice is by placing a coherent light source behind a screen containing a single pinhole. Sample volumes and image sensors placed on the other side of the screen then experience illumination as a point source of coherent light, greatly simplifying downstream interpretation and analysis. To further simplify interpretation of the resulting holographs, the sample volume is typically placed far enough from the pinhole so that the incident light is approximated as a plane wave. A pinhole filter provides a convenient way producing a homogenous source of coherent light. However, pinhole filters also have significant disadvantages. For example, pinhole light sources are energetically inefficient, as only a very small fraction of the generated light (e.g., less than 1%) passes through the pinhole to arrive at the sample volume. Thus, a vast majority of the generated light goes to waste. The pinhole filter itself also adds complexity to the hardware and the overall setup, represents a manufacturing and design constraint, and is a potential point of failure (e.g., due to obstruction of the pinhole by dust).

Advantageously, the use of pinholes can be avoided by applying an algorithmic flat-field correction to the holographs. A flat-field correction process reduces the need for homogenous sources of coherent light, and may thereby avoid the need for a pinhole light source by allowing the use of a coherent source of light (such as a laser diode) to illuminate the sample volume directly. In practice, direct illumination normally yields nonuniform illumination patterns which interfere with interpretation and analysis of the resulting holographs. One way of flattening these non-uniformities is by using a calibration holograph. Calibration holographs may be generated empirically by capturing a holograph without a sample present, or determined based on the laws of diffraction and the physical characteristics of the light source. Flat-field correction may produce an extremely flat profile across the corrected holograph, thereby increasing the signal-to-noise ratio of data extracted from the holograph, such as reconstructed z-plane photographs of objects. The improved signal-to-noise may be relevant to analysis of both the holographs and reconstructed photographs. Field-flattening may also improve the signal-to-noise of cellular growth indicators extracted from the flattened holographs.

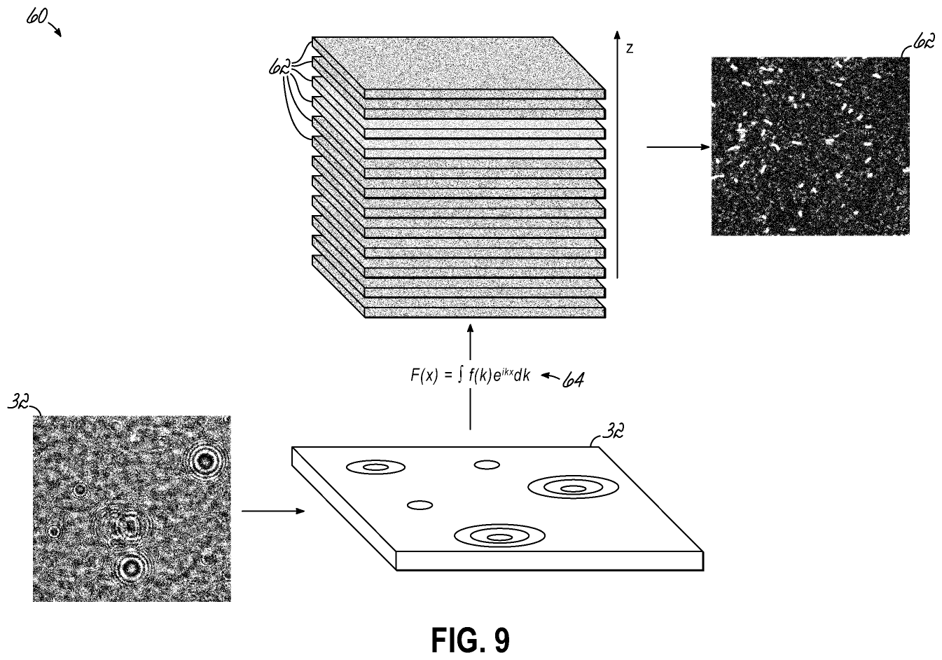

FIG. 9 depicts an exemplary image reconstruction process 60 that extracts photographs 62 from a holograph 32 using a holographic image transformation process such as a Fourier transformation 64. If a lens (not shown) is placed between the image sensor 16 and sample volume 18, different planes within the sample volume 18 located along the z-axis may be brought into focus by adjusting the position of the lens, thereby generating a photographic image at the image sensor 16. A distance u between the z-plane and lens, a distance v between the image sensor 16 and lens, and the focal length f of the lens that will focus a photographic image of a particular z-plane on the image sensor 16 are related by:

$$\frac{1}{u} + \frac{1}{v} = \frac{1}{f} \qquad Eqn . 1$$

Applying the Fourier transform 64 to the holograph 32 provides a focusing function similar to that of a lens, thereby converting the diffraction pattern represented by the holograph 32 into one or more photographs 62 of one or more respective planes along the z-axis.

One method that may be used to track cellular growth in a sample volume 18 is to monitor changes in the variation in the brightness of the holograph 32 across time. These changes in the variation of brightness may provide a cellular growth indicator which can be used to detect cellular growth without reconstructing photographs 62 from the holograph 32. A dispersion factor refers to a factor that has a value of zero if the value of each pixel is the same, and that increases as the values of the pixels become more diverse. Examples of dispersion factors include, but are not limited to, variance, standard deviation, variance-to-mean ratio, range, interquartile range, mean absolute difference, median absolute deviation, and average absolute deviation. Dispersion factors such as standard deviation may be influenced by variations at all length-scales within the holograph 32, including length-scales too long to originate from cells, e.g., length scales greater than 500 μm. Thus, brightness variations at long length-scales in the holograph 32 may add noise to dispersion-based cellular growth indicators and reduce sensitivity. Long length-scale noise may be generated by uneven illumination of the sample volume, variations across the sample container, etc.

Flattening the holograph 32 prior to extracting cellular growth indicators can reduce or eliminate the above described sources of long length-scale noise. Image flattening may be accomplished using the flat-field correction approach described above, by subtracting an nth-order two-dimensional polynomial fit of the image from itself, or by applying high-pass filters to the image, for example. Any of these image flattening approaches can dramatically reduce long length-scale noise. Reducing long length-scale noise may enable detection of cellular growth hours before it would otherwise be possible.

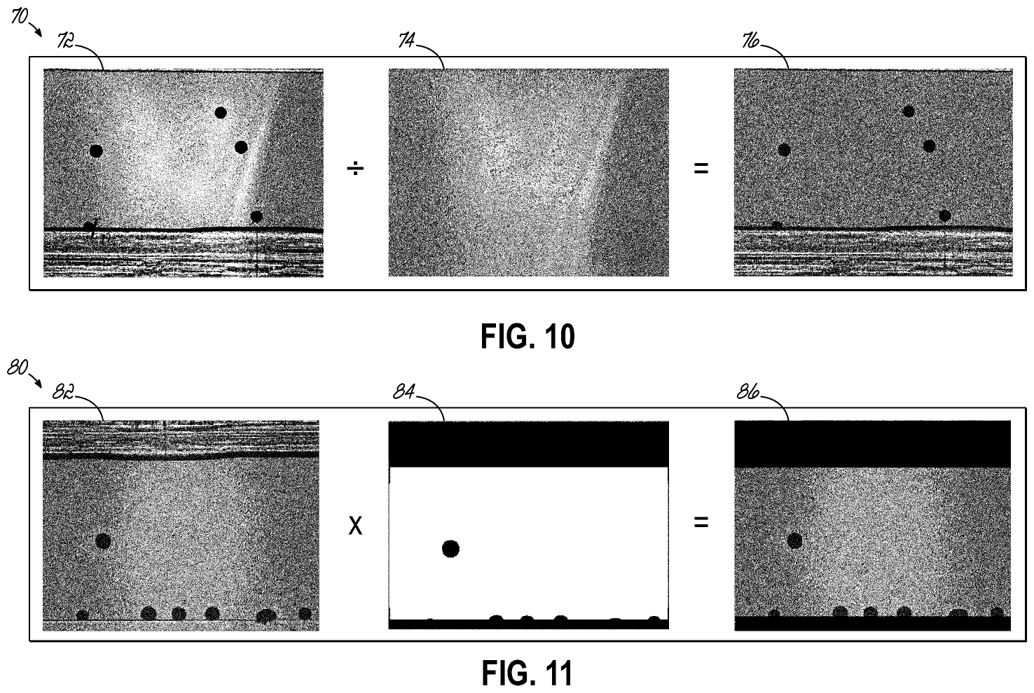

FIG. 10 depicts an exemplary image flattening process 70 for flattening a holograph (e.g., an unprocessed or “raw” holograph 72) using a calibration holograph 74. The calibration holograph 74 may be generated by causing the holographic imager 10 to capture one or more “blank” holographs without a sample container 14 and/or sample volume 18 placed between the light source 12 and image sensor 16. One of these blank holographs may then be used as the calibration holograph 74. In an alternative embodiment in which multiple blank holographs are captured, a plurality of blank holographs may be stacked to generate the calibration holograph 74. A subsequent raw holograph 72 of a sample volume 18 taken by the holographic imager 10 may then be divided by the calibration holograph 74 to generate a flattened holograph 76. As can be seen from the exemplary images of FIG. 10, flattening the raw holograph 72 in this way removes the lighting artifacts visible in the unprocessed holograph 72, which could otherwise interfere with the analysis thereof.

FIG. 11 depicts an exemplary image masking process 80. Masking is another type of image processing that may be used to remove artifacts associated with the experimental setup before extracting sample information from a holograph. Sources of artifacts that may be removed by masking include: (1) geometric features of the sample container 14 and any other portions of the consumable between the light source 12 and image sensor 16, (2) debris objects within the sample holder, on the image sensor 16, or otherwise in a line-of-sight between the light source 12 and image sensor 16, and/or (3) air bubbles in the sample volume 18.

Given the geometric predictability of these objects over time as they appear in holographs, the identification of these objects may be performed based on their time-invariant characteristics. The holograph 82 to be processed may first be analyzed to identify areas of the holograph 82 containing unwanted artifacts. Once these areas are identified, a mask 84 may be defined that covers the unwanted artifacts. The mask 84 may be then applied to the holograph 82 (e.g., by multiplying the holograph 82 by the mask 84) to remove the unwanted areas, and the resulting masked holograph 86 used for sample analysis. Masking may be performed before or after other pre-processing steps, such as flattening.

Sample container geometric features are one type of artifact that can typically be removed from a holograph by masking. For example, the edges of the sample container (or other features associated with the consumable) may appear in raw holographs. These artifacts may be detected as areas of the holograph having a distribution of pixel intensities which is markedly different from that of the areas of the holograph including diffraction patterns. This information, along with the known geometry of the sample container, may be leveraged to mask container geometric features from downstream analysis. In this way, downstream analysis may exclude these interfering features, thereby concentrating cellular growth indicator extraction to regions of the holographs containing information related to the sample volume. This concentration may increase the signal-to-noise ratio of the extracted cellular growth indicators, thereby improving the speed and reliability of cellular growth detection.

Image subtraction may also be performed to remove unchanging objects that can interfere with downstream analyses. A complementary approach to systematically reject unwanted holographic features 34 of a holograph 32 may be through comparison of holographs 32 of the same sample at multiple time points. Biological activity of interest tends to produce holographic features 34 that change with time, while holographic features 34 produced by debris objects and chamber sidewalls tend to remain static. Static holographic features 34 may be systematically excluded from analysis by subtraction of holographs 32 collected at earlier time points from those collected at later time points. Thus, subtraction may be beneficial to direct analysis of holographs without reconstruction. This technique may be effective at removing a set of holographic features 34 associated with the consumable and static objects appearing in the holographs 32.

Another form of interfering holographic features 34 are holographic features 34 caused by objects that are not of interest, but which also change over time. Objects that are not of interest, but which can produce time varying holographic features 34 in a holograph 32 may include, but are not limited to, fibers, macroscopic or microscopic bubbles in aqueous samples, microscopic particles, irregularities in the consumable which scatter light differently across time, etc. Unlike confocal microscopy, these undesirable features do not need to be “in focus” to have a negative impact on the analysis of holographs. Time changing interfering objects 22 can occur within the sample container 14, on top or underneath the sample container 14, or even directly on the image sensor 16. They can typically be subtracted from a holograph 32, but not necessarily from photographs 62 reconstructed from the holograph 32.

Macroscopic debris objects typically cause the areas of the holograph 32 they affect to have a distribution of pixel intensities that is markedly different from unaffected areas of the holograph 32. Affected areas can typically be identified as having an outsized contribution to the tails of what would otherwise be a Gaussian distribution of pixel intensities across the holograph 32. Comparing local pixel intensity distributions in this way is one method to detect and reject debris objects.

Commonly encountered interfering objects are bubbles within an aqueous sample. In addition to generating regions of particularly dark or bright pixel intensities on the holograph 32, bubbles may also be characterized by their round shape. The detection strategies discussed above are often successful in detecting bubbles. Hough Transforms and OpenCV based Blob detection are also sensitive to the round shape of bubbles, and offer a complementary detection mechanism. Embodiments of the processes disclosed herein may employ a combination of one or more of any of the above described approaches to detect and exclude bubbles from downstream analyses.

Microscopic small sized debris produce diffraction patterns similar to those of individual cells, which can make them difficult to differentiate from cells. One way debris objects 22 may be differentiated from cellular objects 22 is by their refractive index. The refractive index of an object 22 may be determined from its diffraction pattern 26, for example, by fitting the holographic feature 34 associated with the object 22 to a pattern generated by an object having known characteristics and/or mathematical formula, or by measuring the phase offset of the object 22 in reconstructed photographs 62.

In confocal microscopy, cells may be identified in images as ‘blobs’ of intensity which stand out from the background. Because of this, total integrated pixel intensity (or mean pixel intensity) scales with cell numbers. Mean pixel intensity thus provides a computationally efficient metric to track cellular growth in confocal microscopy. With in-line holography, objects 22 in the sample volume 18 contribute holographic features 34 to the holograph 32 with intensity variations having sine and cosine functions. The nature of these holographic features 34 is that their integrated intensity (and thus mean intensity) is zero. This is because for every peak in brightness there is also a corresponding trough which cancels out the peak. For this reason, metrics which respond to changes in global pixel intensity are insensitive to the presence or absence of objects 22, and may therefore have limited utility for tracking cellular growth.

Metrics which are better suited for responding to the presence or absence of sines and cosines are those which respond to intensity variation rather than integrated intensity. Thus, the standard deviation—of pixel intensity in a holograph 32 can provide an effective metric for detecting cellular growth. Another metric that may be used to detect cellular growth in holographs 32 is the variance σ² of pixel intensity in the holograph 32, referred to herein as the holographic intensity variance. The holographic intensity variance may also have an additional desirable quality in that it can scale linearly with increasing cell counts. As a result, biological properties such as division rates can be conveniently extracted from growth curves based on the holographic intensity variance of holographs taken over a period of time.

The following is an example of how to utilize holographic intensity variance to determine object concentration. To extract division rates and other relevant parameters, it may be desirable to have a metric that scales with cell concentration, and preferably that scales linearly with cell concentration. Because objects 22 add sinusoidal waves to holographs 32, it makes sense to use metrics to capture that variability in a way which scales linearly with cell concentration. Variance may be defined as:

$${σ}^{2} = \frac{1}{N} {\sum }_{i = 1}^{N} {( {I}_{i} - \overline{I} )}^{2} \qquad Eqn . 2$$

where Iᵢ represents the intensity of pixel i, and Ī represents an average intensity of the set of pixels for which the variance is being determined.

When an object 22 (e.g., a cell) is added to the sample volume 18, the average pixel intensity of a holograph 32 of the sample volume 18 may be unchanged due to the sinusoidal nature of the diffraction pattern 26 produced by the object 22. However, the intensity of individual pixels typically does change due to the additional sinusoidal diffraction patterns 26 added by the object 22. The impact of one cell may be modeled in one dimension for pixels that are infinitely small to allow the use of integrals and continuous functions rather than sums. In this model, variance may be defined by:

$${σ}^{2} = \frac{1}{L} {\int }_{0}^{L} {( I ( x ) - \overline{I} )}^{2} dx \qquad Eqn . 3$$

When an object is added, I(x) will pick up sine terms such as I₁(x)≈I₀(x)+sin(x), where I₀(x) is the intensity before adding the object 22 and I₁(x) is the intensity after adding the object 22. Hence the variance σ² becomes:

$${σ}^{2} = \frac{1}{L} {\int }_{0}^{L} {( I ( x ) + sin ( x ) - \overline{I} )}^{2} dx \qquad Eqn . 4$$

which can be expanded as follows:

$${σ}^{2} = \frac{1}{L} {\int }_{0}^{L} \{ {sin}^{2} ( x ) + ( I ( x ) - \overline{I} ) sin ( x ) + {( I ( x ) - \overline{I} )}^{2} \} dx \qquad Eqn . 5$$

$${σ}^{2} = \frac{1}{L} [ {\int }_{0}^{L} {sin}^{2} ( x ) dx + {\int }_{0}^{L} ( I ( x ) - \overline{I} ) sin ( x ) dx + {\int }_{0}^{L} {( I ( x ) - \overline{I} )}^{2} dx ] \qquad Eqn . 6$$

In Equation 6, the first integral ∫₀^{L} sin²(x)dx≈½× the number of peaks (which is a constant), the second integral ∫₀^{L}(I(x) −Ī) sin(x)dx=0 due to the sine function, and the third integral ∫₀^{L}(I(x)−Ī)²dx is simply the variance σ² before the object was added. Accordingly,

$${σ}_{1}^{2} = \frac{{N}_{peaks}}{2 L} + \frac{1}{L} {\int }_{0}^{L} {( I ( x ) - \overline{I} )}^{2} dx = \frac{{N}_{peaks}}{2 L} + {σ}_{0}^{2} \qquad Eqn . 7$$

Equation 7 defines—at least approximately—how the variance σ² of pixel intensity in a holograph 32 varies when an object 22 is added, and indicates that the holographic intensity variance increases by a constant. Stated mathematically,

$$\frac{\partial {σ}^{2}}{\partial {N}_{object}} ≅ \frac{{N}_{peaks}}{2 L} = constant \qquad Eqn . 8$$

Thus, holographic intensity variance increases linearly as cells are added. Advantageously, the holographic intensity variance can be extracted directly from a series of holographs 32 of sample volumes 18 including growing and dividing cells.

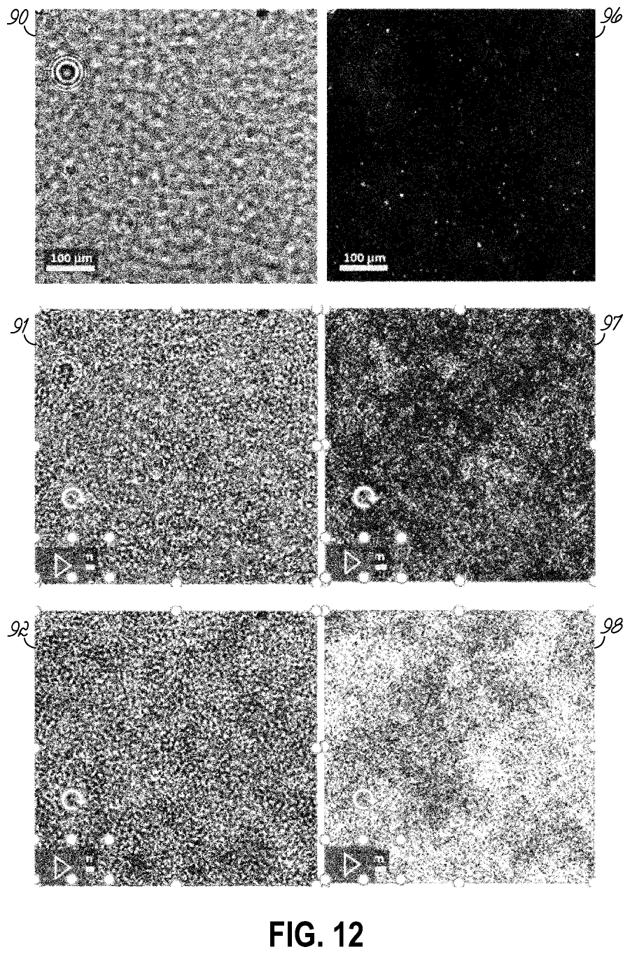

FIG. 12 depicts a sequence of holographs 90-92 and a sequence of photographs 96-98. The holographs 90-92 were generated using light having a wavelength λ=405 nm at 30 minutes (holograph 90), two hours (holograph 91), and four hours (holograph 92) after a sample volume 18 was prepared. Each of the photographs 96-98 was reconstructed from a respective one of the holographs 90-92 for z=266 μm. As can be seen from each sequence of images, there was significant cellular growth in the sample volume 18 over the period. The holographic intensity variance was measured directly from holographic images (not shown) at regular intervals over a five hour period, and compared to the mean pixel intensity P of reconstructed images at the specified z-height in the sample container 14.

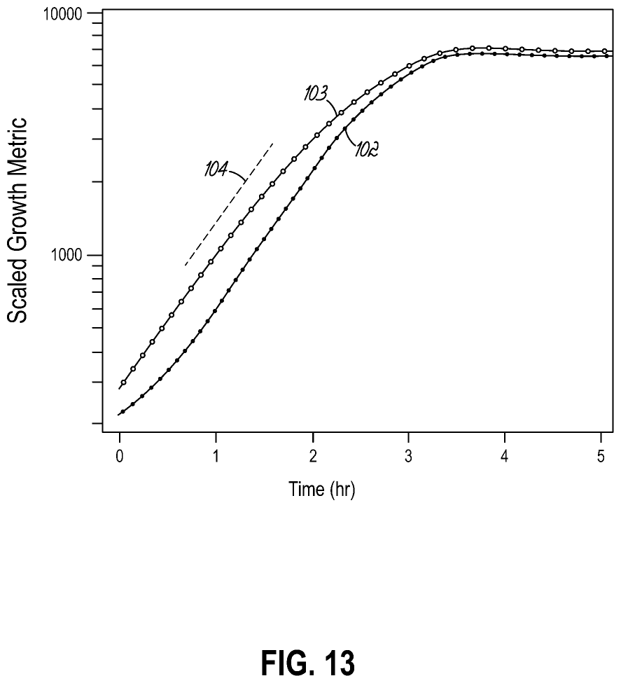

FIG. 13 depicts a graph including a plot 102 of the holographic intensity variance for the holographs described above, a plot 103 the mean pixel intensity P of the reconstructed images generated from those holographs, and a plot 104 showing a slope representing a number of microorganisms multiplying at a growth rate of 1.7 divisions per hour. As can be seen, the growth curves for both the holographic intensity variance of the holographs and the mean pixel intensity P of the reconstructed images are similar in slope and magnitude. Thus, using variance measurements of pixel intensity in a time series of holographs 32 compares favorably with the mean of pixel intensity of reconstructed photographs 62 from the same holographs 32.

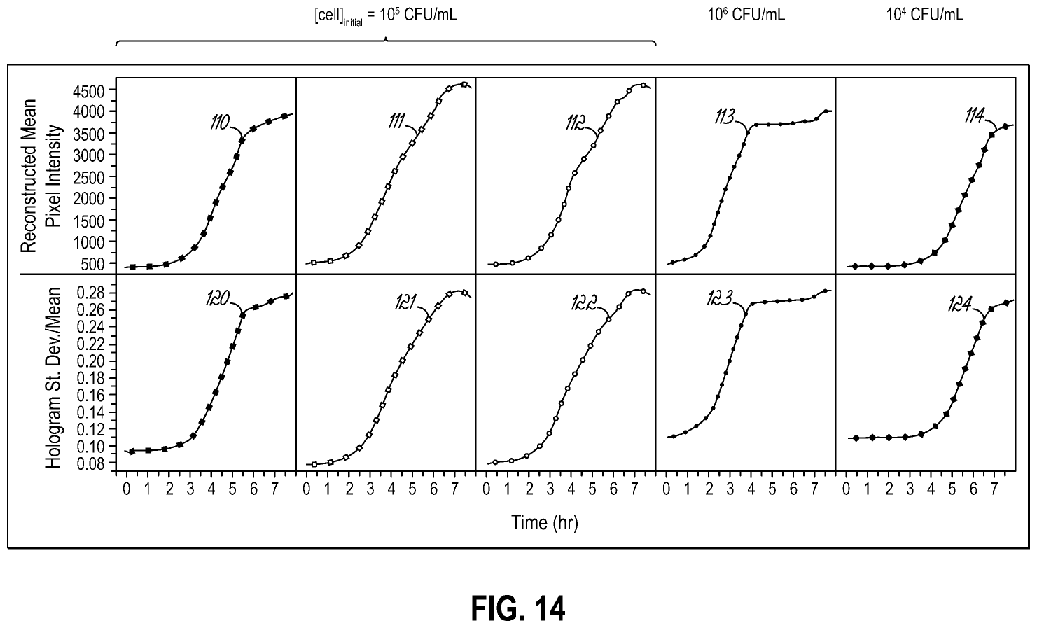

FIG. 14 depicts plots of growth curves for E. coli microorganisms at various cell seeding concentrations. Plots 110-114 show the mean pixel intensity P of reconstructed photographs extracted from the holographs, and plots 120-124 show the standard deviation of the intensity divided by the mean intensity for the holographs of the samples. The cell seeding concentration was 10⁵ CFU/mL for plots 110-112 and 120-122, 10⁶ CFU/mL for plots 113 and 123, and 10⁴ CFU/mL for plots 114 and 124. The normalized standard deviation of the holographic intensity tracks well with the number of microorganisms as they grow and divide, and compares favorably with the mean intensity of the photographs reconstructed from the holographs at the same time points. The shapes of the curves are nearly identical, which indicates that the tracking of the normalized standard deviation of the holographic intensity is a good growth indicator for microbes. Thus, normalized standard deviation of the holographic intensity can provide valuable behavioral data without the need to perform full image reconstruction from the holographs.

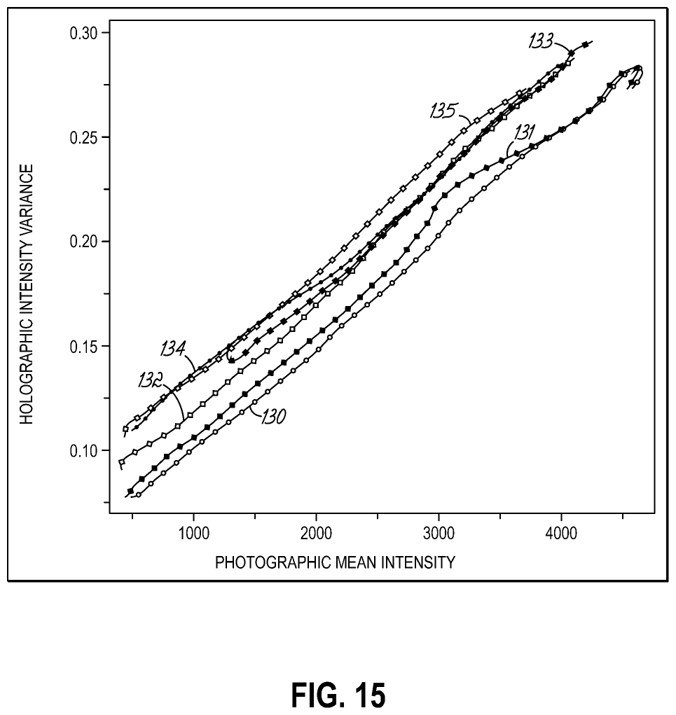

FIG. 15 depicts plots 130-135 of holographic intensity variance 6² versus photographic mean intensity p for each of the measurement scenarios of FIG. 14. The depicted plots demonstrate the linear relationship between tracking object growth by holographic intensity variance as compared to photographic mean intensity.

A more granular way to monitor cellular growth may be provided by detecting each cell individually in the holograph 32. Detecting individual objects 22 may enable the number of objects 22 to be measured directly rather than inferred from a coarser indicator, such as holographic intensity variance. In confocal microscopy, individual objects 22 can be detected by identifying localized blobs of intensity within photographs. This approach may be ineffective with holographs 32 because individual objects 22 contribute sinusoidal holographic features 34 instead of blobs. However, individual objects 22 may be detected in a holograph 32 based on the radial symmetry of the diffraction patterns they generate.

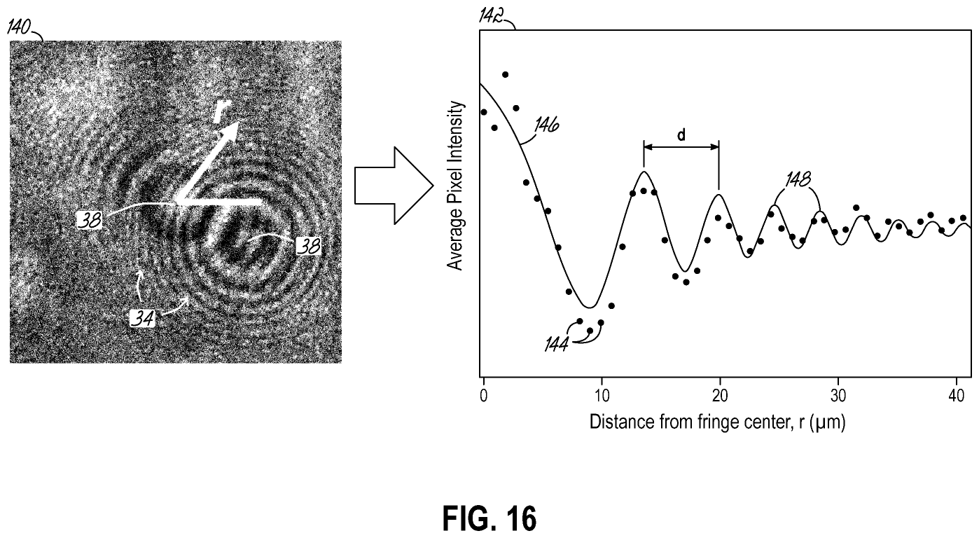

FIG. 16 depicts a holograph 140 including overlapping holographic features 34 and a graph 142 of average pixel intensity verses distance from the center 38 of one of the holographic feature 34. A holographic feature 34 related to a specific object 22 may be analyzed by plotting an average pixel intensity of the holographic feature 34 as a function of distance from its center 38, as shown by datapoints 144. A suitable mathematical formula (e.g., a polynomial or sinc function) may be fitted to the datapoints 144 to facilitate analysis of the holographic feature 34. Exemplary plot 146 depicts a mathematical formula fitted to the datapoints 144 of graph 142, and has a decaying sinusoidal shape including peaks 148. Analysis of holographic features 34 may be used to track the specific behavior of the object 22 generating the diffraction pattern 26 that produced the holographic feature 34 as a function of time. One type of analysis involves examining the amplitude of and distance d between the peaks 148 of a fitted function. The fit constants for generating plot 146 include an image sensor to object distance z_{c} of 249 μm, a signal strength β_{c} of 658 V/μm, a length/of 12.6 μm, and a propagation delay y of light through the object of 5.1 or 292 degrees.

It may be presumed that plane light waves with electric field E_{PLANE} are propagating along the z-axis and incident on the object 22 at a point in space. If the object 22 is infinitesimally small, then light 20 is diffracted around the object 22, but no light 20 passes through the object 22. This creates a spherical wavefront with electric field E_{POINT}. If these two wave sources are allowed to interfere on a plane orthogonal to the z-axis (e.g., the photosensitive surface of image sensor 16), they will interfere according to superposition:

$${E}_{TOTAL} = {E}_{PLANE} + {E}_{POINT} \qquad Eqn . 9$$

To derive the exact interference pattern formed on the plane, one must establish the functional forms of those waves in space. E_{PLANE} is propagating along the z-axis, and can be written as:

$${E}_{PLANE} = {E}_{1} {e}^{ikz} \qquad Eqn . 10$$

E_{POINT} is radiating spherically from a point in space, and can be written as:

$${E}_{POINT} = {E}_{2} {e}^{ik δ - ψ} \qquad Eqn . 11$$

where δ is the distance between the scattering object 22 and another arbitrary point, and ψ is the phase shift imparted on the light passing through the object. The total electric field at any point is space can then be written as:

$${E}_{TOTAL} = {E}_{1} {e}^{ikz} + {E}_{2} {e}^{ik δ - ψ} \qquad Eqn . 12$$

Focusing on the shape of the diffraction pattern 26 and ignoring the magnitude, we can neglect the amplitude component by setting:

$${E}_{0} = {E}_{1} = {E}_{2} \qquad Eqn . 13$$

So, the total electric field can be derived as follows:

$${E}_{TOTAL} = {E}_{0} ( {e}^{ikz} + {e}^{ik δ - ψ} ) \qquad Eqn . 14$$

What gets recorded on the photosensitive surface of image sensor 16 is not the electric field E, but rather the intensity/of the electric field E, which is the square of the electric field:

$$\begin{matrix}I = \vert {E}_{{TOTAL \vert}^{2}} = {E}_{0}^{2} ( {e}^{i k z} + {e}^{i k δ - ψ} ) ( {e}^{- i k z} + {e}^{- i ( k δ - ψ )} ) \\ = {E}_{0}^{2} [ 2 + {e}^{i ( k z - δ )} + {e}^{- i ( k z - δ ) + ψ )} ]\end{matrix} \qquad Eqn . 15$$

Using the hyperbolic trigonometric identity cosh(a)=(e^{a}+e^{-a})/2, Equation 15 can be simplified to:

$$I = 2 {E}_{0}^{2} \{ 1 + cosh [ i ( k z - k δ + ψ ) ] \} \qquad Eqn . 16$$

Note that cosh(0)=(e⁰+e⁰)/2=1, and that sinh(0)=(e⁰−e⁰)/2=0. Applying these to the intensity equation we have:

$$I = 2 {E}_{0}^{2} \{ 1 + cos [ ( k z - k δ + ψ ) ] \} \qquad Eqn . 17$$

From this general expression, a coordinate system can be defined to determine the two-dimensional diffraction pattern 26 recorded by the image sensor 16 as the holographic feature 34 of holograph 140. The coordinate system may be defined such that the sensor plane 30 is orthogonal to and intersects the z-axis at z=0, and the object 22 is located at (0, 0, z), i.e., directly above the origin (0, 0, 0). All locations on the sensor plane 30 can then be expressed as (x, y, 0), and b can be expressed as:

$$δ = \sqrt{{x}^{2} + {y}^{2} + {z}^{2}} \qquad Eqn . 18$$

Substituting Equation 18 into Equation 17 gives:

$$I = 2 {E}_{0}^{2} \{ 1 + cos [ kz - k \sqrt{{x}^{2} + {y}^{2} + {z}^{2}} + ψ ] \} \qquad Eqn . 19$$

Which is the final form of the point scatterer diffraction pattern 26 on the sensor plane 30 of image sensor 16. For practical purposes, it may be convenient to express the wave number k in terms of wavelength λ:

$$k = 2 π / λ \qquad Eqn . 20$$

Due to the rotational symmetry of the diffraction pattern 26, it may also be convenient to use the radial distance from the patterns center, which is provided by:

$${r}^{2} = {x}^{2} + {y}^{2} \qquad Eqn . 21$$

Substituting Equations 20 and 21 into Equation 19 gives:

$$I = 2 {E}_{0}^{2} \{ 1 + cos [ \frac{2 π}{λ} ( z - \sqrt{{r}^{2} + {z}^{2}} ) + ψ ] \} \qquad Eqn . 22$$

which has local maxima and minima whenever the argument of the cosine=nπ, where n=0, 1, 2, . . . .

These extrema locations r_{EXT} can be determined for n=0, 1, 2, . . . and are useful to rapidly determine parameters Z and $ from only the locations of fringe peaks and troughs of the mathematical formula:

$$- \frac{2 π}{λ} ( z + \sqrt{{r}_{EXT}^{2} + {z}^{2}} ) + ψ = \pm n π \qquad Eqn . 23$$

Solving Equation 23 for r_{EXT} yields:

$${r}_{EXT} = \sqrt{{( z + \frac{λ ( ψ \pm n π )}{2 π} )}^{2} - {z}^{2}} \qquad Eqn . 24$$

Diffraction patterns 26 produced by small objects 22 generally comprise a plurality of concentric circles around a center point. Therefore, circle detection approaches can be an effective way of rapidly detecting objects 22 and their individual coordinates from holographs 32. For example, Canny Edge Detection treatment of holographs 32 followed by a Hough Transform may be used to detect circles in a narrow radii range. One strategy to rapidly identify cell-like objects is to use Equation 24 to choose good candidate radii for the search. This approach is computationally efficient, but can also yield false positives. A slower but more robust strategy is to iterate the Hough Transform across a broad range of radii to identify (x, y) locations associated with many circles of various sizes, which is a characteristic of concentric circles centered on (x, y). The presence of concentric circles in a holograph 32 is a reliable indicator of cell-like objects 22 in the sample volume 18.

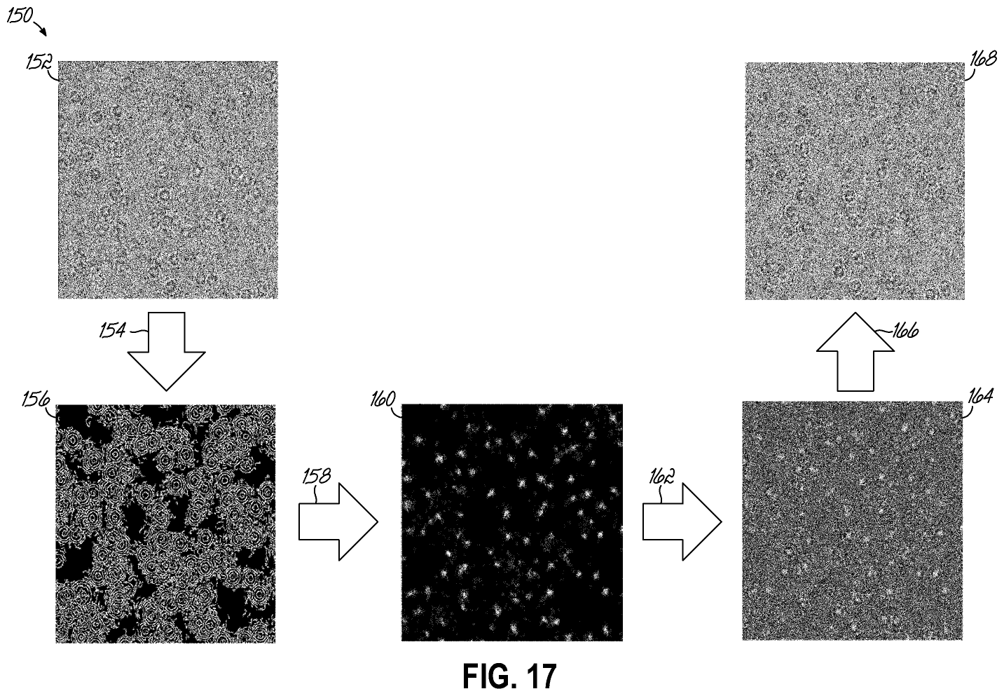

FIG. 17 depicts an exemplary object detection process 150 in which a holograph 152 is first subjected to an edge detection process 154, e.g., Canny Edge Detection. The resulting edge enhanced image 156 may then be subjected to a Hough transformation 158 to produce an accumulation score image 160. The accumulation score image 160 may then be superimposed 162 on the holograph 152 to produce a composite image 164. Holographic features 34 may then be identified 166 on the composite image 164 based on the accumulated scores in the accumulation score image 160 to produce a final image 168, and the holographic features 34 identified in the final image 168 counted.

With the (x,y) coordinates of the objects 22 known, the z-position of each object 22 can be determined based on the shape of the holographic feature 34, which is essentially the same as the shape of the diffraction pattern 26 that generated the holographic feature 34. The z-position of each object 22 may be determined by performing a two-dimensional fit of Equation 22 to each individual holographic feature 34, where z is determined as a free parameter to optimize the fit. A similar and potentially faster process may be to first average the holographic feature 34 over the azimuth angle ϕ in polar coordinates to produce a one-dimensional function in r. A one-dimensional fit may then be performed to Equation 22 to determine the z-position of the object 22. Another potentially fast process may be to average the holographic feature 34 over the azimuth angle ϕ in polar coordinates to produce a one-dimensional function in r, and then use peak detection to determine the radial positions r_{EXT} of the peaks and troughs of the fitted function. The z-position can then be directly calculated from the various r_{EXT} using Equation 25. Advantageously, this approach avoids a curve fitting step.

In an alternative embodiment, the z-position of an object 22 may be determined using geometric triangulation between different light sources 12. For example, by using multiple light sources 12 in which each light source 12 is above a different (x, y) position of the image sensor 16 as shown in FIG. 2. The positions of the light sources 12 may be known beforehand, or may be determined using a calibration step. Having multiple light sources 12 enables differences in the coordinates of the centers 38 of holographic features 34 between the various holographs 32 to be used to determine the height of the object 22. The mathematics behind this involves using the altitude angles (θₗ, θₘ, θₙ) to determine the z-position of the object 22.

As described above, with point objects, diffraction is the only source of scattering, and none of the light 20 is phase shifted by passing through the object 22. However, many objects of interest (e.g., cells) have finite size and allow light 20 to pass through them. Light 20 passing through the object 22 may experience a modified velocity v according to the index of refraction n of the object 22, where v=c/n. This change in velocity as compared to propagation through the suspension media may ultimately manifest as a phase shift y of the light 20 by some amount. This phase shift y can be modeled in the framework of the derivation above using Equation 22, in which the phase shift y appears in the argument of the cosine. It is therefore possible to determine the phase shift y by fitting the mathematical formula of Equation 22 to the holographic feature 34. With the phase shift y determined, it is also possible to estimate the index of refraction n of the object 22, which provides information about the material composition and volume of the object 22. Knowing the index of refraction n for each object 22 may also facilitate distinguishing cells from non-cellular debris.

The processes described above may be applied individually or in any combination to a holograph, or they may be applied to portions of the holograph. These portions of the holograph may comprise subsets of pixels, with each subset defining a contiguous portion of the holograph. Subsets of pixels that form regular shapes within the image (e.g., triangles, squares, or hexagons) may be referred to as “tiles”. The process of tiling a holograph may be referred to as tessellation of the holograph.

Applying these processes separately to one or more portions of a holograph may facilitate detection of local phenomena, which can provide early indicators of bulk activity to follow. For example, some organisms exhibit a heterogeneous response to effectors such as antimicrobial agents, where most of the population dies but a small subset of the population exhibits resistance. In bulk measurements, such as broth microdilution using turbidimetric readings over long time periods, the organism would be correctly identified as resistant because eventually the resistant subpopulation grows to quantities high enough to be macroscopically detectable. However, in fast-timescale measurements, there is the potential to incorrectly characterize the organism as being susceptible to the antimicrobial agent after the majority of organisms are observed to die.

Extracting cellular growth indicators from individual portions of a holograph may increase the likelihood of identifying local resistance. This is due to the increased impact of a small area of resistance on the cellular growth indicators of the portion of the holograph in which the resistance exists. This increase in sensitivity may allow sample volumes to be evaluated over a shorter time scale than would otherwise be possible.

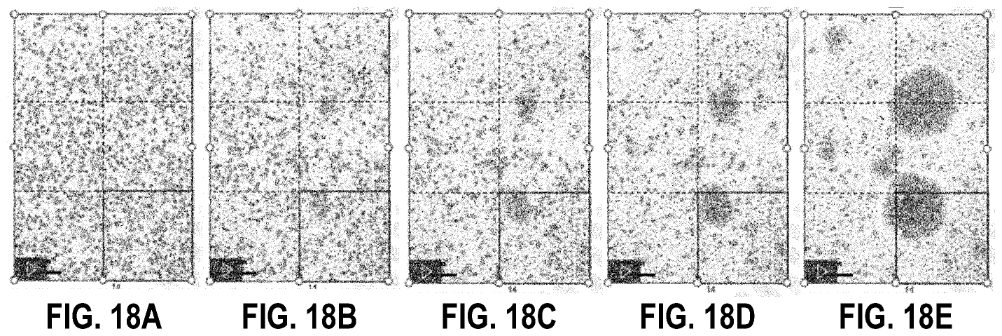

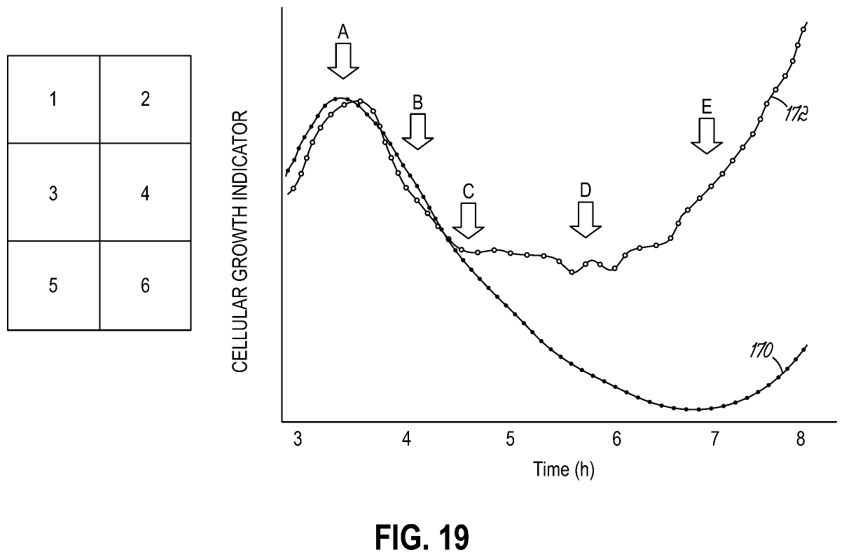

FIGS. 18A-18E depict a sequence of photographs A-E of a sample volume 18, with FIG. 18A depicting the earliest photograph, and FIG. 18E depicting the latest photograph. The photographs A-E show cellular growth of Klebsiella oxytoca (CDCJI 380), which was incubated with 0.5 mg/mL of the antibiotic Meropenem over a period of about eight hours. The photographs A-E provide an example of a complex response of microorganisms to an antibiotic. A holograph 32 of the sample volume 18 was captured every ten minutes during the incubation period. FIG. 19 depicts a graph including a plot 170 of a cellular growth indicator (e.g., the holographic intensity variance) extracted from the full holograph 32, and a plot 172 of the cellular growth indicator extracted from the set of pixels comprising tile six of the holograph 32. The times corresponding to the photographs A-E of FIGS. 18A-18E are indicted by correspondingly labeled arrows in the graph of FIG. 19. Based on the full-holograph plot 170, the microorganism appears to be sensitive to the dose of antibiotic as the cellular growth indicator decreases after three and a half hours of incubation, followed by a slight increase after seven hours of incubation. The slight rise in the cellular growth indicator after seven hours is the earliest indication of a potential resurgence of cell growth, e.g., due to the microorganism becoming resistant.

In contrast, the tile plot 172 of the cellular growth indicator extracted from tile six provides an indication of resurgent growth due to emerging antibiotic resistance at about four and a half hours. The divergence of tile plot 172 from the full-holograph plot 170 begins at about the three and half hour mark. This is due to the response of the microorganism to the antibiotic in the localized region of the sample volume corresponding to tile six. This region of the sample volume contains a subpopulation of bacterial cells that appear to have become resistant to the antibiotic, and have begun to grow in colony clusters. This example of delayed resistance to an antibiotic is clinically relevant, and recognizing the resistance early in a test can lead to timely administration of the correct antibiotic at the right dose to treat the patient. From an algorithmic perspective, systematically dividing holographs into portions (e.g., tiles) and calculating the cellular growth indicator for each portion can be used to identify events or hot spots in the growth or death of microorganisms.

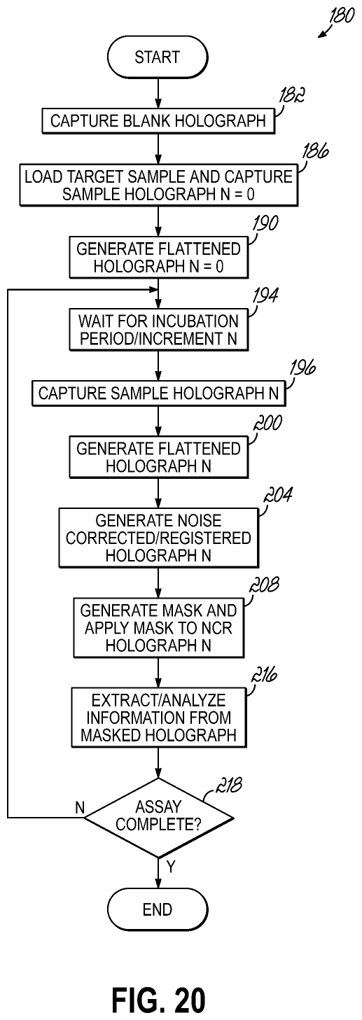

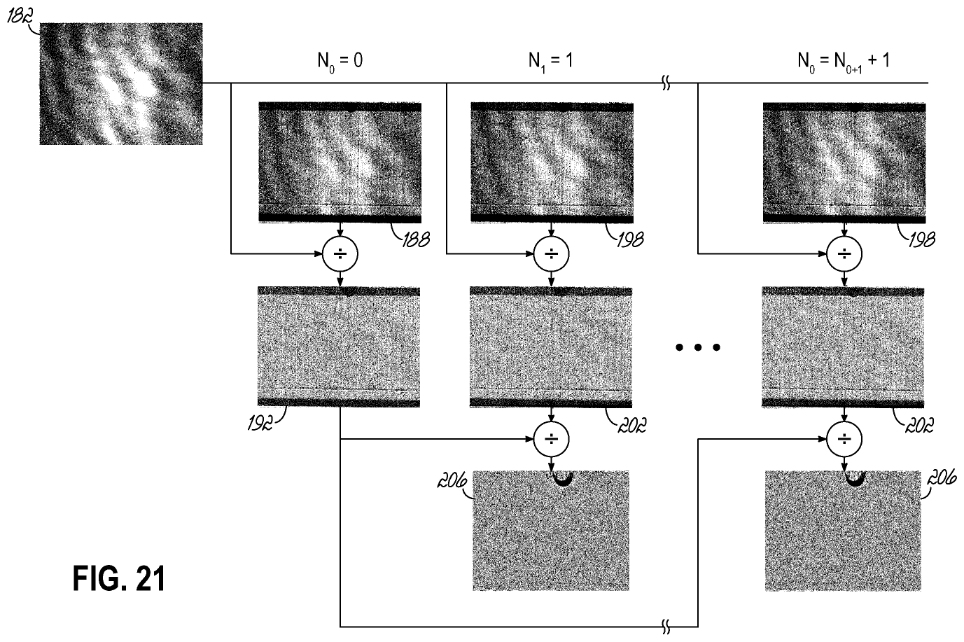

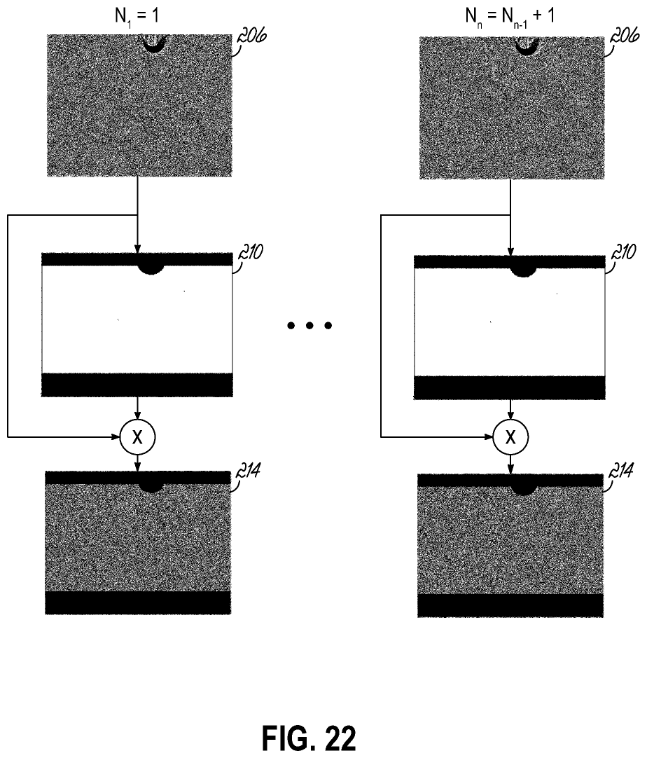

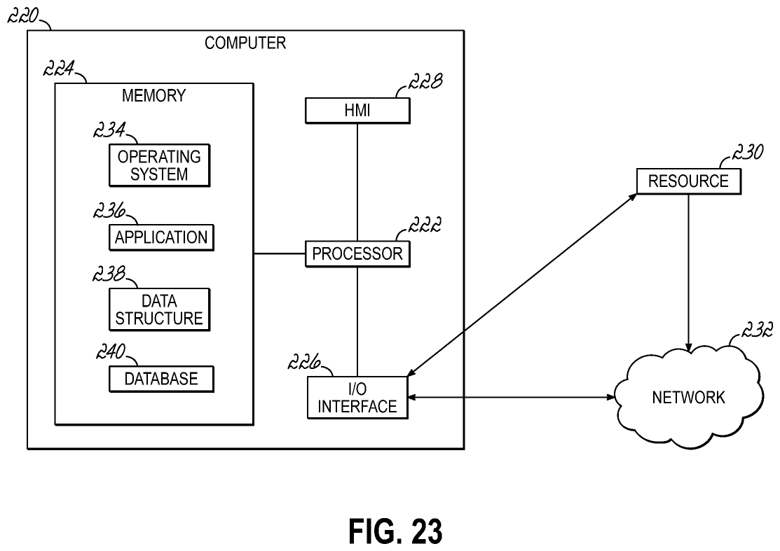

FIG. 20 depicts a flowchart illustrating an exemplary process 178 for performing and/or facilitating the performance of an analytical procedure (e.g., an assay) on a target sample. FIGS. 21 and 22 depict exemplary images that may be captured, generated, and analyzed by the process 178. The process 178 may be implemented, for example, by the sample analysis system 40, or any other suitable system that captures and analyzes holographs of a target sample, such as a sample container 14 containing a sample volume 18.

Referring now to FIGS. 20 and 21, in block 180, the process 178 captures a blank holograph 184. The blank holograph 184 may be a holograph captured without a target sample between the image sensor 16 and light source 12, e.g., before the target sample has been loaded into the sample analysis system 40. In block 186, the process 178 places the target sample (e.g., a sample container 14 containing a sample volume 18) between the light source 12 and image sensor 16. Placement of the target sample may be performed manually or automatically, e.g., by a laboratory technician or robotic sample loading device. After the target sample is in place, the process 178 captures a holograph 188 (e.g., a sample holograph N=0) of the target sample.

In block 190, the process 178 uses the blank holograph 182 to generate a flattened holograph 192 from the sample holograph 188, e.g., by dividing the sample holograph 188 by the blank holograph 182. The blank holograph 182 may have been captured with the same holographic imager 10 as the sample holograph 188, only without the sample volume 18 or consumable in place. Flattening the sample holograph 188 may remove distortions and nonuniform illumination patterns from the sample holograph 188 that are introduced by the sample volume 18 and/or consumable. As compared to the sample holograph 188, the flattened holograph 192 may lack or have reduced background lighting anomalies, reflectance anomalies, and other anomalies that can interfere with image analysis. Holographs taken early in the analytical procedure (e.g., before any alterations to the target sample have occurred) may be used as a reference holograph to reduce noise in later holographs, as described in more detail below.

In block 194, the process 178 may wait for a period of time sufficient to allow the target sample to incubate, and increments N, e.g., N=N+1. A typical incubation period may be between ten minutes and one hour, and can vary depending on the characteristics of the test sample. In block 196, the process 178 captures another sample holograph 198 (e.g., sample holograph N=1), before proceeding to block 200 and generating a flattened holograph 202. The flattened holograph 202 may be generated by dividing the sample holograph 198 by the same blank holograph 182 used to flatten the previous sample holograph 188.

In block 204, the process 178 generates a noise corrected/registered holograph 206 using a previously generated flattened holograph (e.g., flattened holograph 192 generated at N=0) as a reference flattened holograph. The process 178 may generate the noise corrected/registered holograph 206 by subtracting the reference flattened holograph from the present flattened holograph 202, and/or by dividing the present flattened holograph 202 by the reference flattened holograph.

Referring now to FIG. 22, and with continued reference to FIG. 20, the process 178 proceeds to block 208, and generates a mask 210 from the noise corrected/registered holograph 206. The mask 210 may be generated by analyzing the noise corrected/registered holograph 206 using an automated algorithm that detects holographic features in the noise corrected/registered holograph 206 produced by unwanted objects. Each mask 210 may be specific to the noise corrected/registered holograph 206 from which it was generated. Thus, the mask 210 may be configured to remove artifacts in the noise corrected/registered holograph 206 caused by impurities, debris, and other objects that are not part of the target sample, but which may change from one sample period to the next. In block 212, the process 178 generates a masked holograph 214 by applying the mask 210 to the noise corrected/registered holograph 206. It may be desirable to mask out all unwanted aspects of the holograph. These unwanted aspects may include, but are not limited to, holographic features in the holograph caused by air bubbles and chamber boundaries, as well as other holographic features that are not informative to the data analyses.

In block 216, the process 178 may extract information (e.g., dispersion factors, holographic features relating to objects in the sample volume, etc.) from the masked holograph 214 and/or one or more portions thereof as described above. The extracted information may then be used to identify and/or quantify changes in the test sample during the analytical procedure. Methods of data extraction may include, but are not limited to, object growth tracking, edge detection and object counting, and image tiling for hot spot detection. Photographs 62 may also be reconstructed from raw and/or processed holographs 32. These reconstructed photographs 62 may target specific z-planes in the sample volume 18 and/or portions thereof based on the information extracted from the holographs 32.

The masked holograph 214 may be used to determine numerous parameters such as object variance, object number, and/or object concentration. Tiling may also be performed to detect unique events in the masked holographs 214. In the case of microorganisms, detected events may be indicative of cell growth, cell death, and/or other notable object alterations that can be derived. In an alternative embodiment of the process 178, the mask 210 may be applied to the flattened holographs 202 and/or the noise corrected/registered holographs 206, and information extracted from one or more of these masked holographs. It should also be understood that each holograph analyzing process, as well as each step of each holograph analyzing process described herein, can be applied individually, in any combination, and/or in any order to analyze holographs.

Referring now to FIG. 24, embodiments of the invention described above, or portions thereof, may be implemented using one or more computer devices or systems, such as exemplary computer 220. The computer 220 may include a processor 222, a memory 224, an input/output (1/O) interface 226, and a Human Machine Interface (HMI) 228. The computer 220 may also be operatively coupled to one or more external resources 230 via the network 232 or I/O interface 226. External resources may include, but are not limited to, servers, databases, mass storage devices, peripheral devices, cloud-based network services, or any other resource that may be used by the computer 220.

The processor 222 may operate under the control of an operating system 234 that resides in memory 224. The operating system 234 may manage computer resources so that computer program code embodied as one or more computer software applications, such as an application 236 residing in memory 224, may have instructions executed by the processor 222. In an alternative embodiment, the processor 222 may execute the application 236 directly, in which case the operating system 234 may be omitted. One or more data structures 238 may also reside in memory 224, and may be used by the processor 222, operating system 234, or application 236 to store or manipulate data.

The I/O interface 226 may provide a machine interface that operatively couples the processor 222 to other devices and systems, such as the external resource 230 or the network 232. The application 236 may thereby work cooperatively with the external resource 230 or network 232 by communicating via the I/O interface 226 to provide the various features, functions, applications, processes, or modules comprising embodiments of the invention. The application 236 may also have program code that is executed by one or more external resources 230, or otherwise rely on functions or signals provided by other system or network components external to the computer 220. Indeed, given the nearly endless hardware and software configurations possible, persons having ordinary skill in the art will understand that embodiments of the invention may include applications that are located externally to the computer 220, distributed among multiple computers or other external resources 230, or provided by computing resources (hardware and software) that are provided as a service over the network 232, such as a cloud computing service.

The HMI 228 may be operatively coupled to the processor 222 of computer 220 to allow a user to interact directly with the computer 220. The HMI 228 may include video or alphanumeric displays, a touch screen, a speaker, and any other suitable audio and visual indicators capable of providing data to the user. The HMI 228 may also include input devices and controls such as an alphanumeric keyboard, a pointing device, keypads, pushbuttons, control knobs, microphones, etc., capable of accepting commands or input from the user and transmitting the entered input to the processor 222.

A database 420 may reside in memory 224, and may be used to collect and organize data used by the various systems and modules described herein. The database 420 may include data and supporting data structures that store and organize the data. In particular, the database 420 may be arranged with any database organization or structure including, but not limited to, a relational database, a hierarchical database, a network database, or combinations thereof. A database management system in the form of a computer software application executing as instructions on the processor 222 may be used to access the information or data stored in records of the database 420 in response to a query, which may be dynamically determined and executed by the operating system 234, other applications 236, or one or more modules.

In general, the routines executed to implement the embodiments of the invention, whether implemented as part of an operating system or a specific application, component, program, object, module or sequence of instructions, or a subset thereof, may be referred to herein as “program code.” Program code typically comprises computer-readable instructions that are resident at various times in various memory and storage devices in a computer and that, when read and executed by one or more processors in a computer, cause that computer to perform the operations necessary to execute operations or elements embodying the various aspects of the embodiments of the invention. Computer-readable program instructions for carrying out operations of the embodiments of the invention may be, for example, assembly language, source code, or object code written in any combination of one or more programming languages.

The program code embodied in any of the applications/modules described herein is capable of being individually or collectively distributed as a computer program product in a variety of different forms. In particular, the program code may be distributed using a computer-readable storage medium having computer-readable program instructions thereon for causing a processor to carry out aspects of the embodiments of the invention.

Computer-readable storage media, which is inherently non-transitory, may include volatile and non-volatile, and removable and non-removable tangible media implemented in any method or technology for storage of data, such as computer-readable instructions, data structures, program modules, or other data. Computer-readable storage media may further include RAM, ROM, erasable programmable read-only memory (EPROM), electrically erasable programmable read-only memory (EEPROM), flash memory or other solid state memory technology, portable compact disc read-only memory (CD-ROM), or other optical storage, magnetic cassettes, magnetic tape, magnetic disk storage or other magnetic storage devices, or any other medium that can be used to store data and which can be read by a computer. A computer-readable storage medium should not be construed as transitory signals per se (e.g., radio waves or other propagating electromagnetic waves, electromagnetic waves propagating through a transmission media such as a waveguide, or electrical signals transmitted through a wire). Computer-readable program instructions may be downloaded to a computer, another type of programmable data processing apparatus, or another device from a computer-readable storage medium or to an external computer or external storage device via a network.

Computer-readable program instructions stored in a computer-readable medium may be used to direct a computer, other types of programmable data processing apparatuses, or other devices to function in a particular manner, such that the instructions stored in the computer-readable medium produce an article of manufacture including instructions that implement the functions, acts, or operations specified in the text of the specification, the flowcharts, sequence diagrams, or block diagrams. The computer program instructions may be provided to one or more processors of a general purpose computer, a special purpose computer, or other programmable data processing apparatus to produce a machine, such that the instructions, which execute via the one or more processors, cause a series of computations to be performed to implement the functions, acts, or operations specified in the text of the specification, flowcharts, sequence diagrams, or block diagrams.

The flowcharts and block diagrams depicted in the figures illustrate the architecture, functionality, or operation of possible implementations of systems, methods, or computer program products according to various embodiments of the invention. In this regard, each block in the flowchart or block diagrams may represent a module, segment, or portion of instructions, which comprises one or more executable instructions for implementing the specified logical function or functions.

In certain alternative embodiments, the functions, acts, or operations specified in the text of the specification, the flowcharts, sequence diagrams, or block diagrams may be re-ordered, processed serially, or processed concurrently consistent with embodiments of the invention. Moreover, any of the flowcharts, sequence diagrams, or block diagrams may include more or fewer blocks than those illustrated consistent with embodiments of the invention. It should also be understood that each block of the block diagrams or flowcharts, or any combination of blocks in the block diagrams or flowcharts, may be implemented by a special purpose hardware-based system configured to perform the specified functions or acts, or carried out by a combination of special purpose hardware and computer instructions.

The terminology used herein is for the purpose of describing particular embodiments only and is not intended to be limiting of the embodiments of the invention. As used herein, the singular forms “a”, “an” and “the” are intended to include both the singular and plural forms, and the terms “and” and “or” are each intended to include both alternative and conjunctive combinations, unless the context clearly indicates otherwise. It will be further understood that the terms “comprises” or “comprising,” when used in this specification, specify the presence of stated features, integers, actions, steps, operations, elements, or components, but do not preclude the presence or addition of one or more other features, integers, actions, steps, operations, elements, components, or groups thereof. Furthermore, to the extent that the terms “includes”, “having”, “has”, “with”, “comprised of”, or variants thereof are used in either the detailed description or the claims, such terms are intended to be inclusive in a manner similar to the term “comprising”.

While all the invention has been illustrated by a description of various embodiments, and while these embodiments have been described in considerable detail, it is not the intention of the Applicant to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the spirit or scope of the Applicant's general inventive concept.

What is claimed is:

1. A sample analysis system, comprising:
a holographic imager configured to generate a holograph of a sample volume;
one or more processors operatively coupled to the holographic imager; and
a memory operatively coupled to the one or more processors and storing program code that, when executed by the one or more processors, causes the system to:
generate a first holograph of the sample volume at a first time, the first holograph including a first plurality of pixels each having an intensity;
determine a first dispersion factor of the intensity of at least a first portion of the first plurality of pixels; and
determine a property of the sample volume based on a value of the first dispersion factor.

2. The sample analysis system of claim 1, wherein the program code causes the system to determine the property of the sample volume based on the value of the first dispersion factor by comparing the value of the first dispersion factor to a predetermined threshold value.

3. The sample analysis system of claim 1, wherein the program code further causes the system to:
generate a second holograph of the sample volume at a second time, the second holograph including a second plurality of pixels each having an intensity;
determine a second dispersion factor of the intensity of at least a second portion of the second plurality of pixels; and
determine the property of the sample volume based on the value of the first dispersion factor by comparing the value of the first dispersion factor to the value of the second dispersion factor.

4. The system of claim 1, wherein the first portion of the first plurality of pixels is one of a plurality of portions of the first plurality of pixels, and the program code further causes the system to:
determine a second dispersion factor of the intensity of a second portion of the first plurality of pixels, and
determine the property of the sample volume based on the value of the first dispersion factor by comparing the first dispersion factor to the second dispersion factor.

5. The system of claim 4, wherein the program code further causes the system to:
identify a portion of interest in the first plurality of portions;
determine a z-height of an object generating a diffraction pattern in the portion of interest; and
analyze the object.

6. The system of claim 5, wherein the program code causes the system to analyze the object by reconstructing a photograph from the first holograph at the z-height.

7. The system of claim 5, wherein the program code causes the system to identify the portion of interest by:
determining a dispersion factor of the intensity of each portion of the first plurality of pixels to generate a plurality of dispersion factors;
comparing the value of each dispersion factor of the plurality of dispersion factors to one or more values of other dispersion factors of the plurality of dispersion factors; and
identifying the dispersion factor of the portion of interest as an outlier from the plurality of dispersion factors.

8. The system of claim 4, wherein each portion of the plurality of portions of the first plurality of pixels comprises a tile of a plurality of tiles of the first holograph.

9. The system of claim 1, wherein the program code further causes the system to:
apply one or more image modification processes to the first holograph prior to determining the first dispersion factor, wherein the one or more image modification processes do not involve image reconstruction.

10. The system of claim 9, wherein the one or more image modification processes include a flat-field correction process.

11. The system of claim 9, wherein the one or more image modification processes include:
identifying one or more irrelevant portions of the first holograph that are not relevant to quantifying a change in the property of the sample volume;
generating a mask configured to remove the one or more irrelevant portions of the first holograph; and
applying the mask to the first holograph.

12. The system of claim 1, wherein the sample volume includes one or both of a plurality of microorganisms and a plurality of eukaryotic cells of animal or human origin.

13. The system of claim 12, wherein the plurality of microorganisms belongs to a species or class of Gram-negative bacteria, Gram-positive bacteria, or fungi.

14. The system of claim 1, wherein the first dispersion factor is a variance.

15. A method of analyzing a sample volume, comprising:
generating a first holograph of the sample volume at a first time, the first holograph including a first plurality of pixels each having an intensity;
determining a first dispersion factor of the intensity of at least a first portion of the first plurality of pixels; and
determining a property of the sample volume based on a value of the first dispersion factor.

16. The method of claim 15, wherein determining the property of the sample volume based on the value of the first dispersion factor includes comparing the value of the first dispersion factor to a predetermined threshold value.

17. The method of claim 15, further comprising:
generating a second holograph of the sample volume at a second time, the second holograph including a second plurality of pixels each having an intensity;
determining a second dispersion factor of the intensity of at least a second portion of the second plurality of pixels; and
determining the property of the sample volume based on the value of the first dispersion factor by comparing the value of the first dispersion factor to the value of the second dispersion factor.

18. The method of claim 15, wherein the first portion of the first plurality of pixels is one of a plurality of portions of the first plurality of pixels, and further comprising:
determining a second dispersion factor of the intensity of a second portion of the first plurality of pixels; and
determining the property of the sample volume based on the value of the first dispersion factor by comparing the first dispersion factor to the second dispersion factor.

19. The method of claim 18, further comprising:
identifying a portion of interest in the first plurality of portions;
determining a z-height of an object generating a diffraction pattern in the portion of interest; and
analyzing the object.

20. The method of claim 19, wherein analyzing the object includes reconstructing a photograph from the first holograph at the z-height.

21. The method of claim 19, wherein identifying the portion of interest includes:
determining a dispersion factor of the intensity of each portion of the first plurality of pixels to generate a plurality of dispersion factors;
comparing the value of each dispersion factor of the plurality of dispersion factors to one or more values of other dispersion factors of the plurality of dispersion factors and
identifying the dispersion factor of the portion of interest as an outlier from the plurality of dispersion factors.

22. The method of claim 18, wherein each portion of the plurality of portions of the first plurality of pixels comprises a tile of a plurality of tiles of the first holograph.

23. The method of claim 15, further comprising:
applying one or more image modification processes to the first holograph prior to determining the first dispersion factor, wherein the one or more image modification processes do not involve image reconstruction.

24. The method of claim 23, wherein the one or more image modification processes include a flat-field correction process.

25. The method of claim 23, wherein the one or more image modification processes include:
identifying one or more irrelevant portions of the first holograph that are not relevant to quantifying a change in the property of the sample volume;
generating a mask configured to remove the one or more irrelevant portions of the first holograph; and
applying the mask to the first holograph.

26. The method of claim 15, wherein the sample volume includes one or both of a plurality of microorganisms and a plurality of eukaryotic cells of animal or human origin.

27. The method of claim 26, wherein the plurality of microorganisms belongs to a species or class of Gram-negative bacteria, Gram-positive bacteria, or fungi.

28. The method of claim 15, wherein the first dispersion factor is a variance.

29. A computer program product comprising:
a non-transitory computer-readable storage medium; and
program code stored on the non-transitory computer-readable storage medium that, when executed by one or more processors, causes the one or more processors to:
cause a holographic imager to generate a first holograph of a sample volume at a first time, the first holograph including a first plurality of pixels each having an intensity;
determine a first dispersion factor of the intensity of at least a first portion of the first plurality of pixels; and
determine a property of the sample volume based on a value of the first dispersion factor.

30. A sample analysis system, comprising:
a holographic imager configured to generate a holograph of a sample volume;
one or more processors operatively coupled to the holographic imager; and
a memory operatively coupled to the one or more processors and storing program code that, when executed by the one or more processors, causes the system to:
generate a first holograph of the sample volume at a first time, the first holograph including a first plurality of pixels each having an intensity;
extract a first set of holographic features from at least a first portion of the first plurality of pixels that belong to a class of shapes including one or more diffraction patterns each associated with a diffraction of light by an object in the sample volume;
determine a first number of holographic features in the first set of holographic features; and
determine a property of the sample volume based on a value of the first number of holographic features.

31. The system of claim 30, wherein the program code causes the system to determine the property of the sample volume based on the value of the first number of holographic features by comparing the value of the first number of holographic features to a predetermined threshold value.

32. The system of claim 30, wherein the program code further causes the system to:
generate a second holograph of the sample volume at a second time, the second holograph including a second plurality of pixels each having an intensity;
extract a second set of holographic features from at least a second portion of the second plurality of pixels that belong to the class of shapes including the one or more diffraction patterns; and
determine a second number of holographic features in the second set of holographic features,
wherein the program code causes the system to determine the property of the sample volume based on the value of the first number of holographic features by comparing the value of the first number of holographic features to the value of the second number of holographic features.

33. The system of claim 30, wherein the class of shapes includes one or more patterns having a radial symmetry.

34. The system of claim 30, wherein the program code further causes the system to:
determine a phase shift associated with light passing through the object in the sample volume.

35. The system of claim 34, wherein the program code causes the system to determine the phase shift by fitting a mathematical formula to a first fringe pattern generated by the object in the first holograph, and extracting a parameter from the mathematical formula indicative of the phase shift.

36. The system of claim 34, wherein the phase shift of the object is used to distinguish the object from one or more other objects having different phase shifts.

37. The system of claim 36, wherein the object is a cell, and the one or more other objects are debris.

38. The system of claim 36, wherein the object is a first type of cell, and the one or more other objects include a second type of cell.

39. The system of claim 30, wherein the first portion of the first plurality of pixels is one of a plurality of portions of the first plurality of pixels, and the program code further causes the system to:
extract a second set of holographic features from a second portion of the first plurality of pixels that belong to the class of shapes including the one or more diffraction patterns;
determine a second number of holographic features in the second set of holographic features; and
determine the property of the sample volume based on the value of the first number of holographic features by comparing the first number of holographic features to the second number of holographic features.

40. The system of claim 39, wherein the program code further causes the system to:
identify a portion of interest in the plurality of portions of the first plurality of pixels;
determine a z-height of the object generating a diffraction pattern in the portion of interest; and
analyze the object.

41. The system of claim 40, wherein the program code causes the system to analyze the object by reconstructing a photograph from the first holograph at the z-height.

42. The system of claim 40, wherein the program code causes the system to identify the portion of interest by:
extracting a set of holographic features from each portion of the plurality of portions of the first plurality of pixels;
determining a number of holographic features in each set of holographic features extracted from the plurality of portions;
comparing the number of holographic features in each set of holographic features to the number of holographic features in other sets of holographic features; and
identifying the number of holographic features extracted from the portion of interest as an outlier from the number of holographic features in the other sets of holographic features.

43. The system of claim 39, wherein each portion of the plurality of portions of the first plurality of pixels comprises a tile of a plurality of tiles of the first holograph.

44. The system of claim 30, wherein the sample volume includes one or both of a plurality of microorganisms and a plurality of eukaryotic cells of animal or human origin.

45. The system of claim 44, wherein the plurality of microorganisms belongs to a species or class of Gram-negative bacteria, Gram-positive bacteria, or fungi.

46. A method of analyzing a sample volume, comprising:
generating a first holograph of the sample volume at a first time, the first holograph including a first plurality of pixels each having an intensity;
extracting a first set of holographic features from at least a first portion of the first plurality of pixels that belong to a class of shapes including one or more diffraction patterns each associated with a diffraction of light by an object in the sample volume;
determining a first number of holographic features in the first set of holographic features; and
determining a property of the sample volume based on a value of the first number of holographic features.

47. The method of claim 46, wherein determining the property of the sample volume based on the value of the first number of holographic features includes comparing the value of the first number of holographic features to a predetermined threshold value.

48. The method of claim 46, further comprising:
generating a second holograph of the sample volume at a second time, the second holograph including a second plurality of pixels each having an intensity;
extracting a second set of holographic features from at least a second portion of the second plurality of pixels that belong to the class of shapes including the one or more diffraction patterns; and
determining a second number of holographic features in the second set of holographic features,
wherein determining the property of the sample volume based on the value of the first number of holographic features includes comparing the value of the first number of holographic features to the value of the second number of holographic features.

49. The method of claim 46, wherein the class of shapes includes one or more patterns having a radial symmetry.

50. The method of claim 46, wherein the method further includes:
determining a phase shift associated with light passing through the object in the sample volume.

51. The method of claim 50, wherein determining the phase shift includes fitting a mathematical formula to a first fringe pattern generated by the object in the first holograph, and extracting a parameter from the mathematical formula indicative of the phase shift.

52. The method of claim 50, wherein the phase shift of the object is used to distinguish the object from one or more other objects having different phase shifts.

53. The method of claim 52, wherein the object is a cell, and the one or more other objects are debris.

54. The method of claim 52, wherein the object is a first type of cell, and the one or more other objects include a second type of cell.

55. The method of claim 46, wherein the first portion of the first plurality of pixels is one of a plurality of portions of the first plurality of pixels, and the method further comprises:
extracting a second set of holographic features from a second portion of the first plurality of pixels that belong to the class of shapes including the one or more diffraction patterns;
determining a second number of holographic features in the second set of holographic features; and
determining the property of the sample volume based on the value of the first number of holographic features by comparing the first number of holographic features to the second number of holographic features.

56. The method of claim 55, further comprising:
identifying a portion of interest in the plurality of portions of the first plurality of pixels;
determining a z-height of the object generating a diffraction pattern in the portion of interest; and
analyzing the object.

57. The method of claim 56, wherein analyzing the object includes reconstructing a photograph from the first holograph at the z-height.

58. The method of claim 56, wherein identifying the portion of interest includes:
extracting a set of holographic features from each portion of the plurality of portions of the first plurality of pixels;
determining a number of holographic features in each set of holographic features extracted from the plurality of portions;
comparing the number of holographic features in each set of holographic features to the number of holographic features in other sets of holographic features; and
identifying the number of holographic features extracted from the portion of interest as an outlier from the number of holographic features in the other sets of holographic features.

59. The method of claim 56, wherein each portion of the plurality of portions of the first plurality of pixels comprises a tile of a plurality of tiles of the first holograph.

60. The method of claim 46, wherein the sample volume includes one or both of a plurality of microorganisms and a plurality of eukaryotic cells of animal or human origin.

61. The method of claim 60, wherein the plurality of microorganisms belongs to a species or class of Gram-negative bacteria, Gram-positive bacteria, or fungi.

62. A computer program product comprising:
a non-transitory computer-readable storage medium; and
program code stored on the non-transitory computer-readable storage medium that, when executed by one or more processors, causes the one or more processors to:
cause a holographic imager to generate a first holograph of a sample volume at a first time, the first holograph including a first plurality of pixels each having an intensity;
extract a first set of holographic features from at least a first portion of the first plurality of pixels that belong to a class of shapes including one or more diffraction patterns each associated with a diffraction of light by an object in the sample volume;
determine a first number of holographic features in the first set of holographic features; and
determine a property of the sample volume based on a value of the first number of holographic features.
